(19) United States
(12) Patent Application Publication
(10) Pub. No.: US 2007/0213590 A1
(43) Pub. Date: Sep. 13, 2007

(54) APPARATUS AND METHODS FOR EXAMINING, VISUALIZING, DIAGNOSING, MANIPULATING, TREATING AND RECORDING OF ABNORMALITIES WITHIN INTERIOR REGIONS OF BODY CAVITIES

(75) Inventor: John B. Squicciarini, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(73) Assignee: GYNTEC MEDICAL, INC., Aliso Viejo, CA (US)

(21) Appl. No.: 11/750,232

(22) Filed: May 17, 2007

Related U.S. Application Data
(63) Continuation-in-part of application No. 11/225,381, filed on Sep. 12, 2005.
(63) Continuation-in-part of application No. 10/938,688, filed on Sep. 10, 2004.
(60) Provisional application No. 60/608,810, filed on Sep. 10, 2004.
(60) Provisional application No. 60/510,706, filed on Oct. 9, 2003.

(51) Int. Cl. A61B 1/06

(52) U.S. Cl. 600/172

(57) ABSTRACT

A portable multi-functional endoscopic device and method for use in the examination of tissue within a corporeal orifice to permit diagnostic, therapeutic or anatomical assessment data to be transmitted, recorded, or analyzed. The device includes a base unit sized and configured to be held in a human hand to permit functional and directional control of the device, an interchangeable head assembly sized and configured to be inserted into the orifice being removably connectable to the base unit, and an inflatable tissue stabilizer disposed external to a distal end of the device. In preferred aspects, the endoscopic device has an image sensor, light source, lens, air pump, and working tools.

### CROSS-REFERENCE TO RELATED APPLICATIONS

This application claims the benefit of priority as a Continuation-in-part of U.S. application Ser. No. 11/225,381, filed on Sep. 12, 2005, which claims priority from U.S. Provisional application No. 60/608,810, filed on Sep. 10, 2004, all of which are incorporated by reference in their entireties, herein. This application also claims the benefit of priority as a Continuation-in-part of U.S. application Ser. No. 10/938,688, filed on Sep. 10, 2004, which claims priority from U.S. Provisional application No. 60/510,706, filed on Sep. 9, 2003, all of which are incorporated by reference in their entireties, herein.

### BACKGROUND OF THE INVENTION

1. Field of the Invention

The field of the invention is endoscopic devices. In particular, a small, portable, light-weight endoscopic device for a mammalian orifice or cavity is presented. In various embodiments, the cavities include, but are not limited to, a vagina, uterus, anus, urethra, nostril, nose, ear canal, and mouth. Certain embodiments of the endoscopic device include a video probe to record video and/or images of the internal cavity area. Further, the endoscopic device includes mechanisms which can be used in conjunction with the visualization technology to vastly improve the accuracy and precision in conducting examinations or procedures, such as taking precise biopsies or by which fluids, tissue, and/or other samples can be accurately collected with minimal contamination for later laboratory analysis. Certain embodiments of the endoscopic device further include an inflatable and/or mechanical tissue stabilizer to expand and open and stabilize an orifice or cavity to improve a range and depth of view of the tissue therein.

There are numerous endoscopic devices known in the art, and the specific use will at least in part determine the specific configuration of the endoscope. However, and regardless of the particular use of the endoscope, heretofore known devices will typically fall within one of two general categories. In one category, the light source and/or the camera is coupled to the flexible endoscope at the handle or controller that is located outside of the person being examined. In the other category, the light source and/or the camera is coupled to the flexible endoscope at the terminal portion that is advanced into the patient. Depending on the particular use, the body of the endoscopic device may be rigid or flexible, and movement of the flexible portion is typically effected via a hand-held controller. Thus, the light source and/or the camera are either on a distal and/or on a proximal end. Consequently, configuration flexibility is typically not achieved with known endoscopes, and a change in a procedure will often necessitate a change of endoscopic device during the procedure. Therefore, while there are numerous endoscopic devices and methods known in the art, all or almost all of them suffer from one or more disadvantages. Thus, there is a need for an improved endoscopic device.

Various embodiments of endoscopic systems, devices and methods of the present invention can be used for examining, visualizing, diagnosing, manipulating, treating and recording of abnormalities with interior regions of body cavities. For example, disclosed herein is an endoscopic device for penetrating, illuminating and taking video images of a human cavity. In one non-limiting application embodiment, an endoscopic device is used to identify and evaluate lesions, infections, warts, melanoma, sexually transmitted disease indicators, and other normal or abnormal features located within a female's reproductive system.

2. Description of the Related Art

There are numerous endoscopic devices known in the art, including for example, a typical endoscope described in U.S. Pat. No. 5,421,339 to Ramanujam et al. In that patent the endoscope comprises a laser with fiber optics carrying light to a probe, and collection fibers carrying induced and reflected light to from the probe to an external sensor. Among other uses described in the '339 reference, the endoscope is used for spectroscopic methods to improve predictive value of colposcopy. While such configurations can have various advantages in stationary use, the light source and image analysis system required for such systems often prevent mobile use.

To render a colposcope more suitable for mobile use, the endoscopic device can be configured to have a head with camera, light emitters, infusion, and suction channels as described in U.S. Pat. App. No. 2002/0022764 to Smith et al., wherein the camera can be located at the end of the probe, or along the side of the probe. Light emitters (or a fiber optic light bundle) can be mounted directly on the end of the head, and a hand held display can be employed in such devices.

Alternatively, as described by Kirsner in U.S. Pat. App. No. 2004/0068162, a device can be configured to enable a patient to perform a colposcopic or other endoscopic self-examination at minimal discomfort, wherein the device transfers the diagnostic information wirelessly to a medical professional. Such endoscopic devices are particularly suitable where frequent self-examination in a private environment is desired. Unfortunately, Kirsner's device fails to provide any test results to a patient, and only advises the patient whether to see the physician or not. Moreover, implementation of Kirsner's device to detect early signs of cervical cancer is hindered by the fact that the sensor appears to be on side of the shaft. Such sensor position typically prohibits generation of a perspective view as the sensor is positioned adjacent the tissue being examined.

Thus, while numerous compositions and methods for endoscopic devices are known in the art, all or almost all, suffer from one or more disadvantages. Therefore, there is still a need for improved endoscopic devices.

### SUMMARY OF THE INVENTION

This document describes various devices and methods for examining, visualizing, diagnosing, manipulating, treating and recording of abnormalities with interior regions of body cavities. For example, disclosed herein is an endoscopic device for penetrating, illuminating and taking video images of a human cavity. In various embodiments, the cavities include, but are not limited to, a vagina, uterus, anus, urethra, nostril, nose, ear canal, and mouth.

In one embodiment, an endoscopic device for the examination of tissue within a corporeal orifice to permit diagnostic, therapeutic or anatomical assessment includes a base unit and an interchangeable head assembly. The base unit is sized and configured to be held in a human hand to permit functional and directional control of the device. The base unit has a proximal end and a distal end. The interchangeable head assembly is sized and configured to be inserted into the orifice. The interchangeable head assembly is removably connectable to the distal end of the base unit. The interchangeable head assembly is detachably linked to the base unit in one or more of either a mechanical, electrical, optical or fluid fashion. An inflatable tissue stabilizer is disposed on an exterior surface of the interchangeable head assembly.

In another embodiment, the present invention is directed to configurations and methods for a visualization device having a head with a lens. Contemplated devices further include an elongated body having a shaft and extendable arms, and a frame that extends the arms outward from the body. A space is configured between the shaft and the arms such that when the arms are extended, the shaft is moveable without necessarily displacing the arms.

In some embodiments of the present invention, a lens is mounted on the head, and a camera and a light emitter can be disposed in the head and/or the body. Especially preferred heads have a base with a substantially round outer boundary, wherein a tissue stabilizer (or “stabilizing ring”) includes extendable arms which are configured to retract to provide a substantially continuous outer boundary with the base. While not limiting to the inventive subject matter, the extendable arms can comprise a first, a second, and a third arm. Additionally or alternatively, the frame extends the arms outward from a retracted position about the body to an extended position farther displaced from the shaft, and the device can further include a working tool (e.g., Pap smear collector, an ultrasound emitter, or a fluid line for a cryogenic fluid, dye, or lavage fluid) deployable from a storage position that is at least partially in a location between the shaft and at least one of the arms. Contemplated devices will further include a connector that carries a signal from the camera to a monitor or other visualization or recording device to vastly improve the accuracy and precision in conducting examinations or procedures, such as taking biopsies or taking precise tissue samples.

In one aspect of the inventive subject matter, a visualization probe includes a head capped with a lens, behind which are functionally mounted a camera and a plurality of light emitters, an internal power source, and a connector that carries a video signal to an external monitor. In particularly preferred devices, the light emitters comprise at least three diodes disposed about an aperture of the camera, most preferably wherein at least two of the light emitters produce light of significantly different colors from one another. Thus, in most preferred devices, it should be recognized that the light passes to the camera without use of fiber optics. In still further contemplated aspects, the connector includes a radio transmitter.

Additionally, or alternatively, contemplated devices include a shaft, and at least one tool (e.g., Pap smear collector, fluid line, or an ultrasound emitter) is disposed in a storage position adjacent the shaft. An elongated stabilizing arm can be moveably disposed between a retracted position close to the shaft and a deployed position wherein at least part of the arm is distanced from the shaft.

In another embodiment, an endoscopic device includes a hand-held base, a camera connected with the hand-held base, and a light source connected with the hand-held base for illuminating an area above the camera. The device further includes a lens structure coupled to the hand-held base and positioned proximate the camera and the light source. A removable, disposable enclosure of the lens structure includes a cylindrical body formed of a rigid, transparent material, a proximal end for being removably coupled to the hand-held base, and a distal end that terminates in a rounded transparent tip.

This application claims the benefit of priority as a Continuation-in-part of U.S. application Ser. No. 11/225,381, filed on Sep. 12, 2005, which claims priority from U.S. Provisional application No. 60/608,810, filed on Sep. 10, 2004, all of which are incorporated by reference in their entireties, herein. This application also claims the benefit of priority as a Continuation-in-part of U.S. application Ser. No. 10/938,688, filed on Sep. 10, 2004, which claims priority from U.S. Provisional application No. 60/510,706, filed on Sep. 9, 2003, all of which are incorporated by reference in their entireties, herein. Additional disclosure relating to endoscopic visualization devices pertaining to balloon dilators and electrical tissue stimulation device is available in U.S. application Ser. No. 11/348,976, filed on Feb. 6, 2006, which claims priority from U.S. Provisional application No. 60/650,060, filed on Feb. 4, 2005, all of which are incorporated by reference in their entireties, herein.

Various objects, features, aspects and advantages of the present invention will become more apparent from the accompanying drawings along with the following detailed description of preferred embodiments of the invention. The details of one or more embodiments are set forth in the accompanying drawings and the description below. Other features and advantages will be apparent from the description and drawings, and from the claims.

### BRIEF DESCRIPTION OF THE DRAWINGS

These and other features, embodiments, and advantages of the present invention will now be described in connection with preferred embodiments of the invention, in reference to the accompanying drawings. The illustrated embodiments, however, are merely examples and are not intended to limit the invention.

FIG. 1 is a schematic vertical cross sectional view of an exemplary embodiment of a device in collapsed configuration according to the inventive subject matter.

FIG. 2 is a schematic view of the device of FIG. 1 in extended configuration.

FIG. 3 is a schematic view of the device of FIG. 2 with the head in retracted position.

FIG. 4 is a schematic view of the device of FIG. 3 in which an exemplary tool is extended through the head.

FIG. 5 is another schematic view of the device of FIG. 1 in use as a colposcopic device.

FIG. 6 is another schematic view of the device of FIG. 2 in use as a colposcopic device.

FIG. 7 is another schematic view of the device of FIG. 3 in use as a colposcopic device.

FIG. 8 is another schematic view of the device of FIG. 4 in use as a colposcopic device.

FIG. 9 shows an endoscopic device in accordance with an exemplary embodiment.

FIG. 10 shows an endoscopic device having a hand-held base and a lens structure in accordance with an exemplary embodiment.

FIG. 11 depicts exemplary tips and enclosures thereof.

FIG. 12 depicts coupling of the tip enclosures in FIG. 11 to an endoscopic device.

FIG. 13 illustrates a power supply and other controls of a device.

FIG. 14 illustrates various tip enclosures.

FIG. 15 depicts an integrated tip enclosure, light source and camera.

FIG. 16 illustrates a tip enclosure extension.

FIG. 17 shows a tip enclosure and flexible extended optic fiber extension.

FIG. 18 shows a device with a tip enclosure having a flexible fiber optic extension.

FIGS. 19 and 20 illustrate various angles of view in accordance with exemplary embodiments.

FIGS. 21 and 22 shows a tip enclosure in accordance with an alternative embodiment.

FIGS. 23-25 illustrate a tissue stabilizing dilator mechanism and uses thereof in combination with an endoscopic device.

FIG. 26A illustrates a schematic, perspective view of an endoscopic device with an inflatable tissue stabilizer according to one embodiment of the present invention.

FIG. 26B illustrates a schematic, partially exploded perspective view of the endoscopic device of FIG. 26A according to one embodiment of the present invention.

FIG. 27 illustrates a schematic cross-section view of the endoscopic device of FIG. 26A according to one embodiment of the present invention, with a tip enclosure attached to a main probe.

FIG. 28 illustrates a schematic, sectional perspective view of embodiments of a lens, light source, and image sensor disposed within an interchangeable head assembly according to one embodiment of the present invention.

FIG. 29 is a schematic, partially exploded perspective view of an embodiment of a main probe of an endoscopic device with a portion of the base unit open to reveal the interior of the device.

FIG. 30 illustrates a schematic, partially exploded perspective view of the tip enclosure of the endoscopic device of FIG. 26A with the inflatable tissue stabilizer moved.

FIGS. 31A-31D illustrate some of the steps in one embodiment of a method for using any of the disclosed embodiments of an endoscopic device with an inflatable tissue stabilizer disposed on a tip enclosure with an optional exemplary working tool.

FIGS. 32A-32D illustrate a side schematic of an embodiment of a shielded working tool, where the shielded working tool is a sleeved sample collection device.

Throughout the figures, the same reference numerals and characters, unless otherwise stated, are used to denote like features, elements, components or portions of the illustrated embodiments. In certain instances, similar names may be used to describe similar components with different reference numerals which have certain common or similar features. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF THE PREFERRED EMBODIMENT

As should be understood in view of the following detailed description, this application is primarily directed to apparatuses, systems and methods for examining, visualizing, diagnosing, manipulating, treating and recording of abnormalities with interior regions of body cavities. As used herein, “distal end” may be understood to apply to a distal region, and is not necessarily limited to a distal surface or a distal tip, but can refer to the entire region near a distal end. Likewise, “proximal end” can be treated in a similar manner.

### 1. Multi-Functional Video Scope Embodiments

The inventor has discovered that an endoscopic device can be configured to allow mobile, office or home use of the device in situ in multiple configurations, and especially to allow examination of a tissue using a plurality of perspective positions of the optical head relative to the examined tissue while maintaining the device in substantially the same position. Contemplated devices further advantageously provide a mechanism that temporarily stabilizes and/or displaces tissue from the optical field that would otherwise obstruct optical examination using a device without that mechanism.

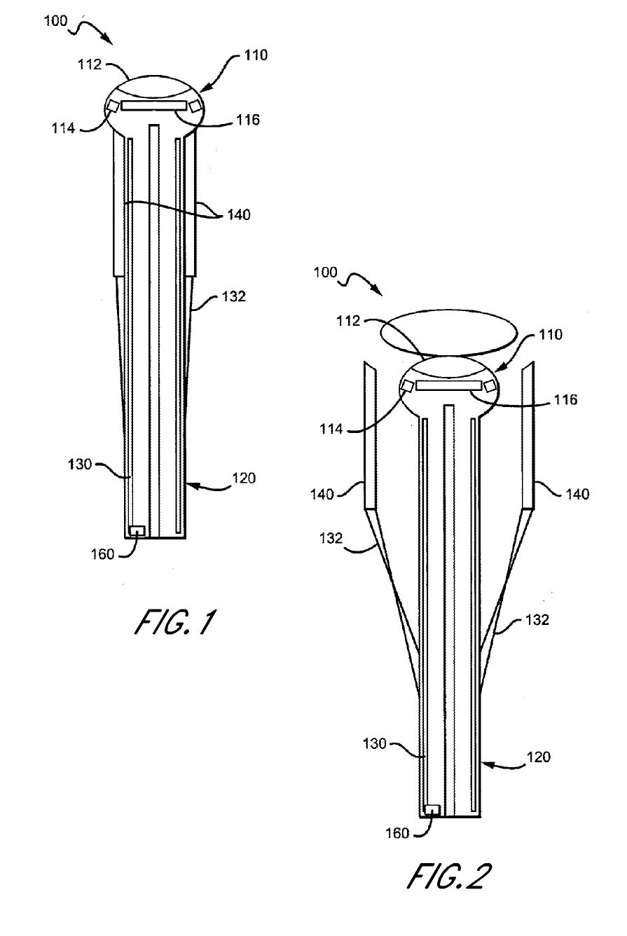

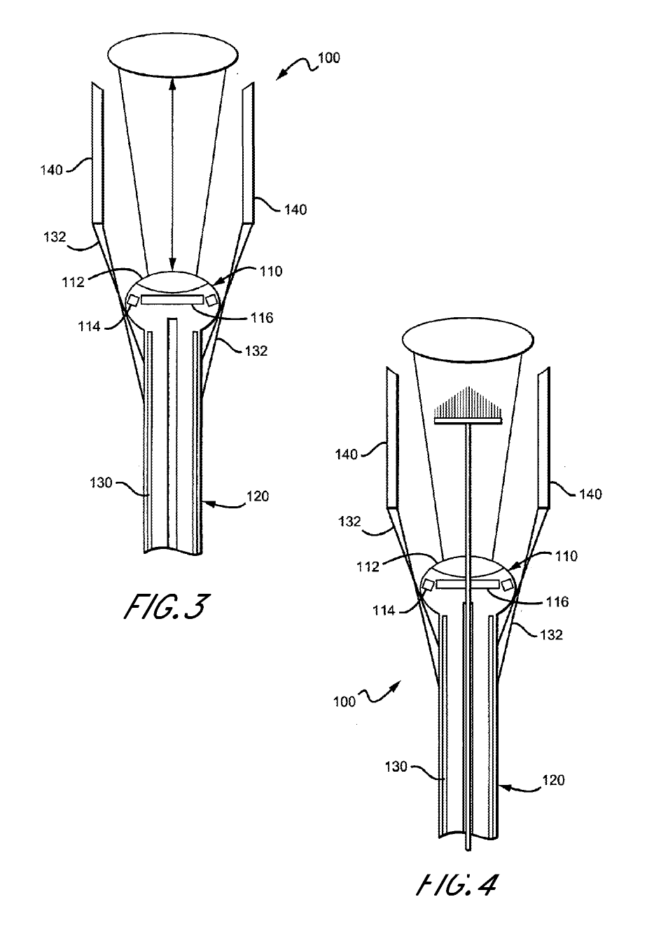

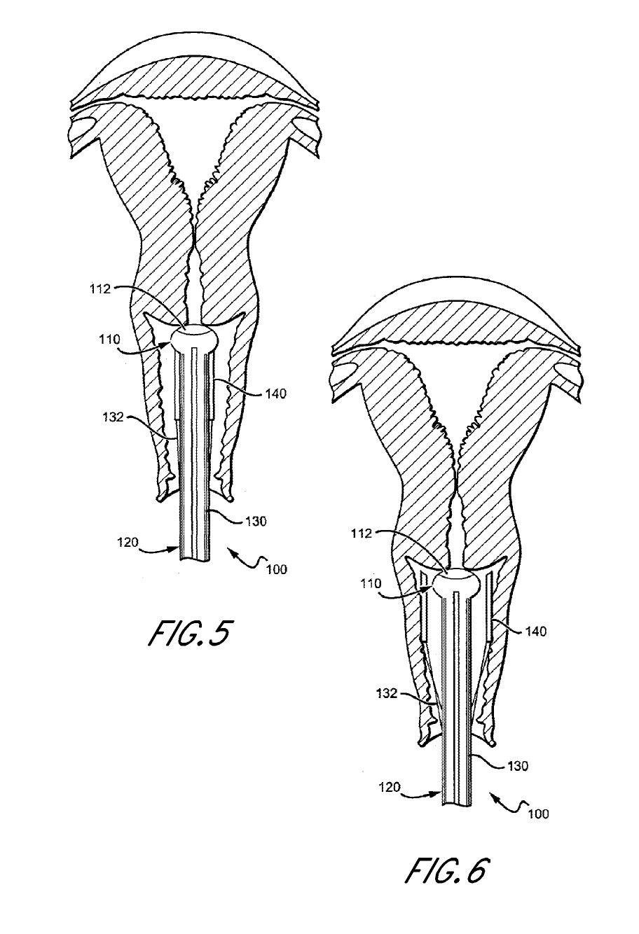

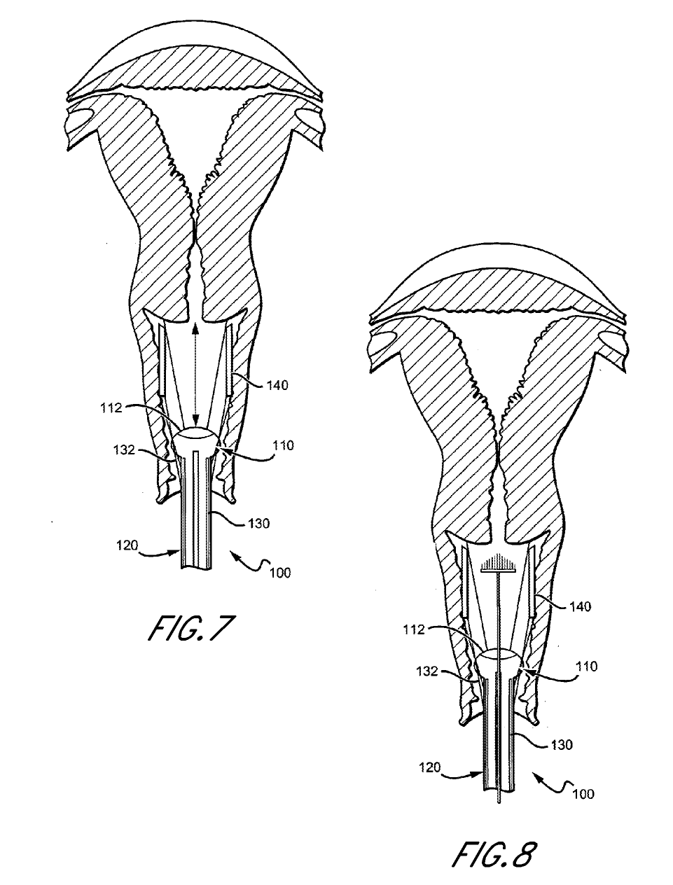

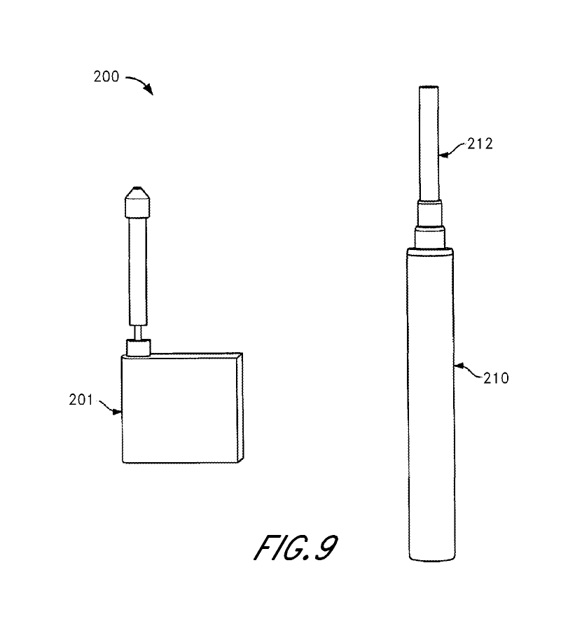

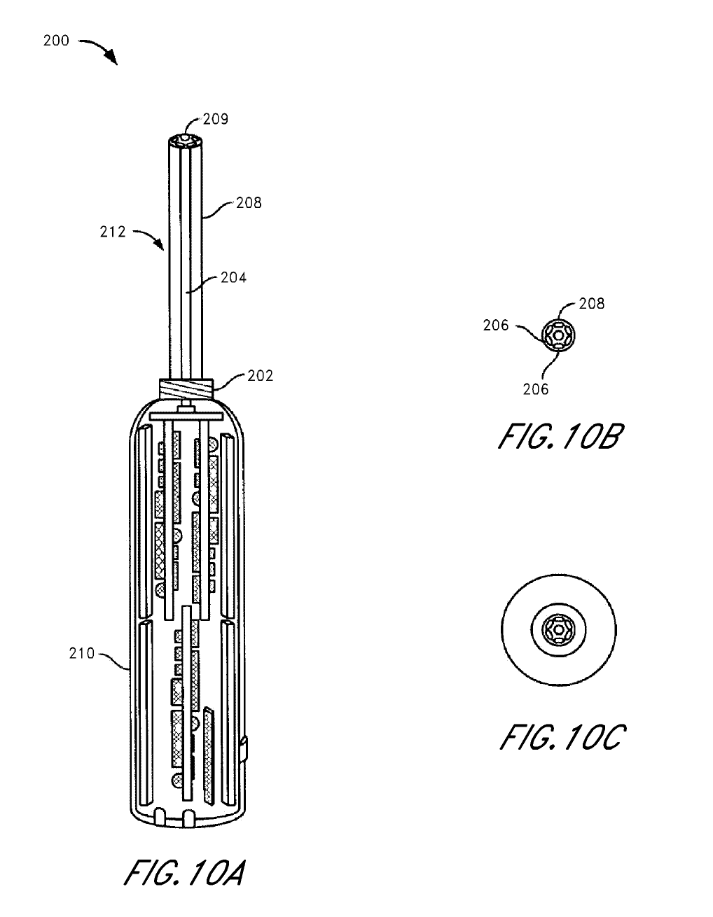

An exemplary endoscopic device 100 in a first configuration (collapsed) is depicted in FIG. 1 in which an interchangeable head assembly, or head 110 having a lens 112, is coupled to a base unit, or shaft 120. Light source 114 and an image sensor, or camera 116, are further disposed within the head 110, but may also be located outside of the device where the device is disposable and coupled to a hand-held base (the light and/or image is transferred through the device to the base via one or more light guides). In one embodiment, coupled to the shaft 120 is a tissue stabilizer frame 130 (which may also be called a stabilizing ring frame, having tracks as indicated), wherein extendable arms 140 (only two are shown, but other numbers of extendable elements) are coupled to the frame 130 via connectors 132 that move within the tracks. Working tools (not shown here, see FIG. 4) are disposed within a space formed between the shaft and the arms, and connector 160 provides video signals from the camera 116 to an imaging device. Alternatively, where the imaging device is disposed in a hand-held base, the connector 160 can be omitted (not shown). FIG. 2 depicts the device of FIG. 1 in a second (extended) configuration in which the head is proximal to a target tissue, while in FIG. 3 the head is moved in a distal position relative to the target tissue without moving the endoscopic device in situ (without repositioning the arms relative to the adjacent tissue). It should be noted that using such configuration, the viewing angle and/or field can be continuously changed without changing the position of the endoscopic device in situ. FIG. 4 depicts deployment of a tool from the device. FIGS. 5-8 depict the device of FIGS. 1-4, respectively, in use as a colposcopic device. With respect to the elements in FIGS. 2-8 and other subsequent figures, the like numerals depict like elements as depicted in FIG. 1. In one embodiment, portions of the devices illustrated in FIGS. 1-8 are disposable covers that can be inserted over the scope tip or attached as a detachable or interchangeable head assembly.

In one embodiment of the inventive subject matter, a contemplated device will include an interchangeable head assembly with a lens, and an elongated body comprising a base unit (or shaft) and one or more extendable arms. In such devices, a frame is coupled to at least one of the arms and configured to extend the arm or arms outward from the body, wherein a space is provided between the shaft and the arm or arms such that when the arms are extended, the shaft is moveable without necessarily displacing the arms.

Depending on the particular use of the endoscopic device, it should be recognized that the interchangeable head assembly can have various sizes and configurations. However, it is generally preferred that the interchangeable head assembly is round or rounded, and more preferably at least partially ovoid or spheroid. Viewed from another perspective, the interchangeable head assembly can be advantageously configured such that the device is advanced accurately and with great precision specifically to the target tissue without injury to adjacent tissue. Furthermore, the shape of the interchangeable head assembly is at least partially depending on the lens and/or optical system that is preferably disposed in the head. Most typically, the head will have a diameter of less than 2 inches, more typically of less than 1 inch, and most typically of less than ½ of an inch. In preferred configurations, the head has a portion that engages with at least one arm to retain the arm in a retracted position (typically where the arm has a minimum distance to the shaft), wherein the retention can be achieved using a movable retention member, or simply by engaging the arm in a corresponding indentation or other non-movable structure. Thus, contemplated heads will include those having a base with a substantially round outer boundary, wherein the extendable arms retract to provide a substantially continuous outer boundary with the base.

In further preferred aspects, the interchangeable head assembly is continuous with the base unit shaft and coupled to the shaft in a removable manner. While not limiting to the inventive subject matter, the shaft has a diameter that is preferably less than that of the head to accommodate (among other elements) the frame and the arms. Depending on the use of the endoscopic device, the shaft can have a diameter of less than 1 inch, more typically of less than ¾ of an inch, and most typically of less than ½ of an inch, while the length of the shaft can be between 2 inches and 20 inches, and even longer. Similarly, the shaft can be configured to be flexible (e.g., passively via deformation, or actively via guide mechanism) where the endoscope is used as a colonoscope, or rigid where the endoscope is used as a laparoscope or colposcope. Furthermore, in various embodiments, the cylindrical head of the disposable portion and the changeable frequency of color illumination and the ability to inject fluids or dyes, and create a suction to collect fluid or tissue samples provides inherent colposcopic functionality.

In one embodiment, the interchangeable head assembly of contemplated devices will include a lens that can further include additional optical components to allow focusing, zooming, and/or change of an angle of visual inspection relative to a hypothetical axis that is parallel to the shaft. For example, and depending on the particular use of the endoscope, the lens can be an integral part of the head in some devices, while in other devices the lens or optical unit is removably placed into the head. With respect to a particular focusing arrangement, it should be recognized that the lens and/or optical system can have a fixed focus (and with that a fixed magnification) or a variable focus and variable magnification. There are numerous such optical devices and configurations known in the art, and all of such devices and configurations are deemed suitable for use herein.

Where the focus is fixed, it is especially preferred that the tip of the device can contact the tissue to be examined to thereby provide the highest magnification. Alternatively, the focal depth may be larger, for example at least ¼ inch, more preferably at least ½ inch, and most preferably 1-20 inches. In such devices, and depending on the particular use, the preferred fixed magnification is between 1 time (or less) to about 10 times (or higher). On the other hand, where the focus can be adjusted, focal depth can be less than ¼ inch, and more typically less than one-eighth of an inch. In such devices, and depending on the particular use, the preferred fixed magnification is between 133 times (or less) to about 20 times (or higher). Furthermore, it should be recognized that contemplated devices can also include a second lens and/or optical system to allow stereoscopic imaging. For the second lens and/or optical system, the same considerations as provided above apply. Additionally, it is contemplated that the lens and/or optical system can include one or more filters (e.g., to filter out excitation light reflected from a tissue, or to provide a narrow band of excitation light).

In one embodiment, the lens and/or optical system is further configured such that the viewing angle can be continuously changed from a coaxial to an angled position. Such configuration is particularly advantageous where the target tissue is not directly in front of the lens/optical system, but offset to one side where a conventional video scope would not be able to acquire an image without altering its configuration (e.g., by bending the tip). For example, suitable angles will include those between 0 (zero) degrees and 60 degrees (and even more), more preferably between 0 (zero) degrees and 45 degrees, and most preferably between 0 (zero) degrees and 30 degrees.

In one embodiment it is generally contemplated that the interchangeable head assembly and/or base unit (or body) may include an image sensor, or camera, and a light emitter. There are numerous cameras suitable for endoscopic use known in the art, and all of them are contemplated herein. For example, especially preferred cameras include a video chip that not only registers light of a wavelength between about 400 nm to about 720 nm, but also near-UV (between 350 nm and 400 nm), UV (less than 350 nm), near IR (between 720 nm and 780 nm), and IR (more than 780 nm) light. Suitable video chips can provide the data directly to a connector that carries the video signal from the camera to a computer, printer, personal digital assistant (PDA) and/or monitor, or can provide the data to a processor that is also disposed in the shaft or head. Data transfer to the processor, computer, and/or monitor can be conventional (e.g., using an USB port or other conventional technology or system) or wireless (e.g., using Bluetooth technology or other wireless technologies or systems).

Alternatively, in another embodiment of the inventive subject matter, at least one of the camera and the light source is disposed in a device (most preferably hand-held) to which the body and head are removably coupled. In such devices, it should be appreciated that the curvature of the lens is parallel to, or even part of the head, and that the lens is optically coupled to the camera via a fiber optic arrangement (or other light guide). Viewed from another perspective, the lens may be an integral part of the head and the light collected by the lens is transferred to the camera (which is disposed in the device that is removably coupled to the body) via fiber optic. Preferably, the light emitter in such devices is also disposed in the device that is removably coupled to the body, and the light is provided to the head via a plurality of optic fibers or other light guides. Most preferably, the light is delivered to the area to be examined in a homogenous manner to allow visualization of an evenly illuminated field.

Thus, it should be recognized that the head and body (together with the frame and optional tools) may be configured as a detachable unit (and in one embodiment as a unit that is disposed after single use) that can be removed from a device that includes the camera and/or light source. In such configurations where the tissue stabilizer (or stabilizing ring) is in an arm configuration, the arms are preferably actuated using a mechanism that is coupled to the body but not to the device that includes the camera and/or light emitter. Various embodiments of a tissue stabilizer are disclosed herein, including stabilizing rings, various arm configurations, and various inflatable membrane configurations. With respect to the arm configurations of the device, it is generally preferred that the device includes a plurality of arms that are coupled to the shaft such that the arms move between a first position and a second position, wherein the arms are proximal to the shaft in the first position and distal to the shaft in the second position. There are numerous manners possible in which the arms can be moved from the first to the second position, and all known manners are deemed suitable for use herein. For example, the arms can be moved by a plurality of actuators that are coupled to the shaft, wherein the actuators are moved by one or more elements (e.g., hydraulic, mechanic, or electric) at least partially disposed in the shaft. More preferably, however, the arms are moved by a frame that is coupled to the outside of the shaft, wherein the frame comprises a plurality of actuators (e.g., using umbrella type actuation) that will move the arms from the first to the second position. There are numerous manners of such actuation known in the art, all of which are deemed suitable for use herein. For example, the frame can have a set of guide rails that extend longitudinally along the shaft, wherein the frame is coupled to the shaft. One end of a first actuator can slidably engage with the guide rail while the other end can engage with one portion of an arm. A second actuator can move the first actuator along the guide rail while a third actuator engages with the second actuator to provide additional moving force. Alternatively, a plurality of actuators can be threaded through the shaft and perpendicularly exit the shaft to connect with the arms or arms. Advancing the actuators in such devices into the shaft will move the arm or arms outwardly.

In one embodiment, the endoscopic device has at least three arms that are circumferentially disposed on the shaft, and that are coupled to the shaft via a frame. The frame preferably extends the arms outward from a retracted position about the body to an extended position farther displaced from the shaft. In still further alternative aspects, the arms may also be moved via a screw-motion.

Depending on the particular configuration of the frame, shaft, and/or tissue stabilizer comprised of arms, it is contemplated that the endoscopic device further includes a working tool deployable from a storage position that is at least partially defined by a space between the shaft and at least one of the arms. For example, where the endoscopic device is a colposcopic device, suitable working tools include a pap smear collector, a fluid line for collecting or dispensing fluids, an air line, Doppler, and/or an ultrasound emitter. Further contemplated devices can also have one or more fluid lines that carry a cryogenic fluid, a dye, and/or a lavage fluid. In another example, where the endoscopic device is a colonoscope, suitable working tools can include a deployable scissor, a cauterizing loop, a needle for injection of pharmaceutical compositions or other uses, and other working tools and devices.

In another aspect of the inventive subject matter, contemplated devices can also include a head that is capped with a lens, behind which are functionally mounted a camera and a plurality of light emitters. An internal power source can provide power to the camera and/or the light emitters, and a connector carries a video signal to an external monitor (with respect to the configuration of the head and coupling to the shaft, the same considerations as provided above apply).

In one embodiment, the light emitters comprise at least three diodes disposed about an aperture of the camera, wherein at least two (and more preferably three) of the light emitters produce light of significantly different colors from one another (i.e., with wavelength maximum at least 20 nm apart). In another embodiment, one or more red-green-blue or RGB light source(s) in which one diode can be digitally controlled to provide all the needed color outputs through control of light frequency can be used. Alternatively, light sources other than a light diode can be used, and suitable alternative light sources include incandescent, laser, and electro-luminescent light sources. Depending on the particular requirements, the light source can be disposed behind a filter that modifies the spectral characteristics of the light source, or can be transmitted via a light guide. However, in one embodiment the light from the light source passes to the camera without use of fiber optics or other light guides. As the camera in such devices is located in the head, it is contemplated that supporting electronic devices can be positioned in the shaft, and more preferably outside the endoscopic device. Consequently, it should be recognized that such devices will include an interface that transfers the image data (processed or raw) to an imaging device. Preferably, such an interface will comprise a connector, and most preferably a wireless connector (e.g., using a radio transmitter, radiofrequency, infrared, microwave, Bluetooth, or other wireless communication format or media).

It is still further contemplated that the devices in which the head includes the camera and a plurality of light emitters have a shaft (preferably as described above), and that at least one tool is disposed in a storage position adjacent the shaft. Furthermore, it is also preferred that the shaft is coupled to a frame and one or more elongated stabilizing arms that are moveable between a retracted position close to the shaft, and a deployed position distal to the shaft. With respect to the various frame configurations that provide movability of the arms to extend outwardly from the body and the movability of the head relative to the target tissue while the device is in situ, the same considerations as discussed above apply.

### 2. Additional Video Scope Embodiments

Disclosed further herein is an endoscopic device with embodiments that can be configured such that an interchangeable head assembly or tip is removably coupled to a hand-held base unit. Embodiments of systems, devices, and methods may be similar to the systems, devices and methods disclosed elsewhere in this patent specification, and may have different or additional features as disclosed herein. For example, in one embodiment of an endoscopic device of the present invention, a fiber optic or other light guide portion in the tip receives light from a light source in the hand-held base. An image sensor, such as a camera or video chip, in the hand-held base receives image information (e.g., reflected and/or emitted light from the tissue to be examined) via a fiber optic or other light guide portion in the tip. In some embodiments, the distal end of the tip includes a lens, which may further be detachable from the tip. In some embodiments, the tip and/or lens is preferably disposable or formed of a disposable material such as plastic or acrylic. In an exemplary embodiment, the hand-held base unit includes the camera, the light source, the image processor, the power supply, and a data interface to relay the image signal to a monitor or other display device.

One embodiment of the image sensor is a camera comprising a charge coupled device (CCD) chip. In various embodiments, the CCD chip may have a size of ¼, ⅓, or ⅙ inch or other similar or smaller sizes, with one embodiment using 400,000 total pixels, and scanned at a rate of 60 Hz using 400 lines. Other embodiments include 2 mega-pixels, 3 mega-pixels, 5 mega-pixels, 6 mega-pixels, 7 mega-pixels and other higher resolution cameras and chips, including high-resolution devices. Suitable light sources include LED and incandescent light sources. Light filters (optical or electronic) can also be used in order to generate a light source of particular, predetermined light characteristics (i.e. luminescence, brightness, etc.). In one embodiment, illumination by the light source is at least 1 lux. Image processing to produce an NTSC image can be accomplished using electronic components, and freeze-frame and continuous output can be provided.

The power supply is preferably included in the hand-held base and includes a rechargeable battery (Li-ion or otherwise). The hand-held base can further include data interfaces for transmission of the CCD signal, and the video signal from the image processor can be output via a wireless interface (Bluetooth, microwave, infrared, etc.) and/or a wired interface (USB, USB2.0, Firewire, or any other hard-wired means).

FIG. 9 depicts an endoscopic device 200 according to an exemplary embodiment of the present invention. The endoscopic device 200 delivers high-resolution, preferably wireless video reproduction and transmission of detailed examinations of body cavities. The device 200 includes a hand-held base unit 210 and an interchangeable head assembly tip 212. The tip 212 includes a lighting source (not shown in this figure) and an image collection mechanism (not shown in this figure), preferably a video camera. The device 200 can be combined with a video receiver 201, such as a microwave or other wireless video receiver, to receive video signals from the device 200. The video receiver 201 can be connected to a monitor, a television, a computer, a tape recorder or other digital video recorder or display device.

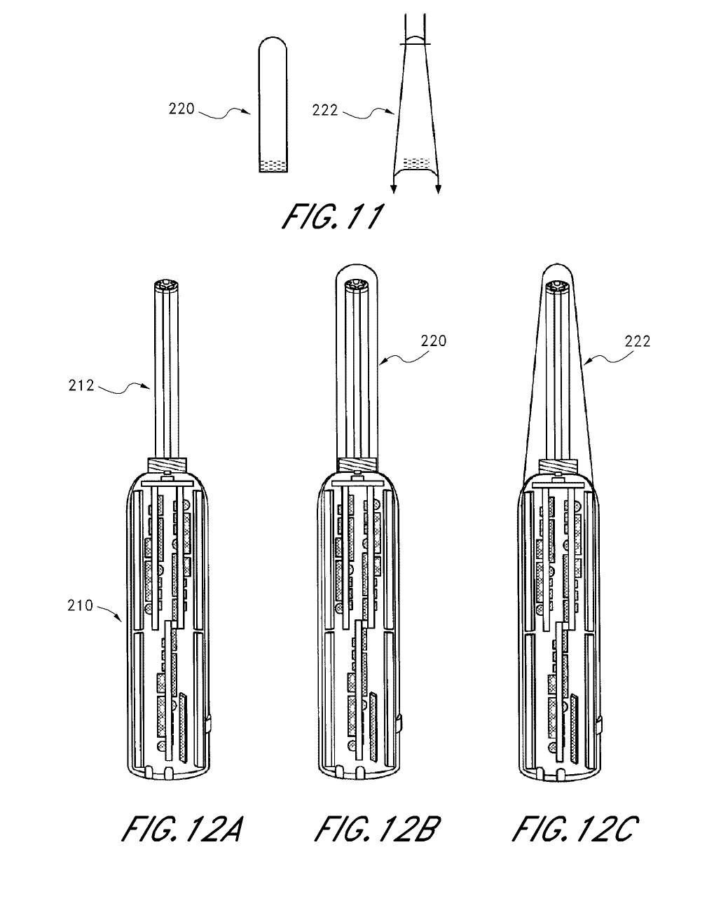

FIGS. 10A-10C depict various views of an endoscopic device 200 in which a camera 202, a light source 206, and other electronic components are disposed in the hand-held base unit 210 and projected through a lens structure 204 disposed within a lens enclosure 208. The lens enclosure 208 may also be considered a tip enclosure, an interchangeable head assembly enclosure, or a disposable optic and sampling component. The lens structure 204 can magnify images or image signals being received by the camera 202, and/or control light from the light source 206 to illuminate tissue being examined. Alternatively, the camera 202 and light source 206 can be disposed within the lens enclosure 208, and controlled by circuitry in the hand-held base 210.

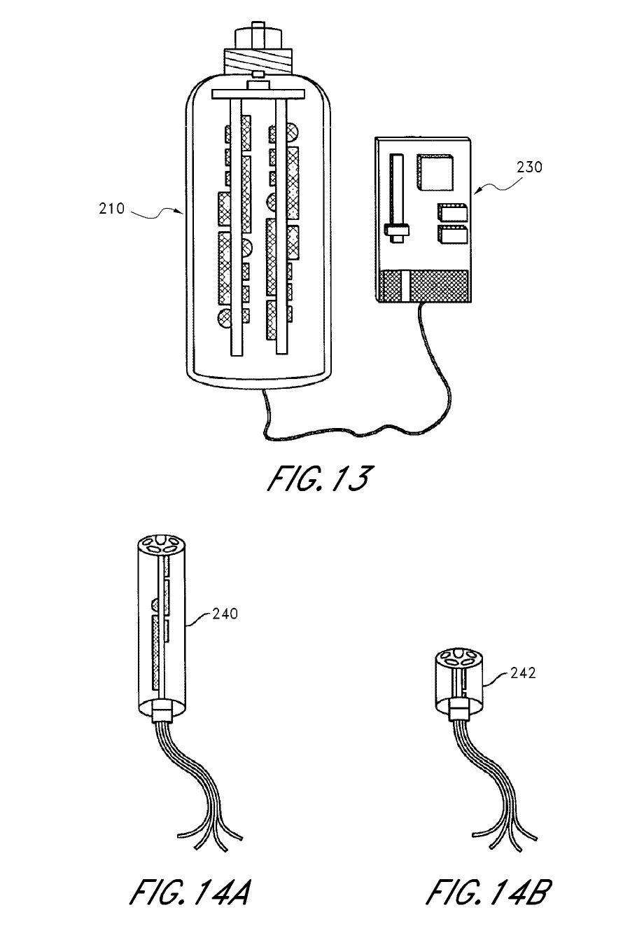

FIG. 11 depicts various embodiments of exemplary interchangeable head assembly tips and enclosures thereof. The illustrated embodiment of tip enclosure 220 is generally cylindrical with a circular, oblong or oval cross-section, and tip enclosure 222 is generally conical and can have a circular, oblong or oval cross-section. The inner diameter of the tip enclosure 220, 222 can be just large enough to fit over a lens extension of an endoscopic device. The tip enclosure 220, 222 can be formed of plastic, Teflon-coated plastic, glass, or Teflon-coated acrylic or other partially or completely transparent materials or coatings that prevent or minimize the adhesion of blood or any other fluids or contaminants. The tip enclosures also serve to spread open a cavity of the patient being examined, to allow full video capture and illumination without being blocked by body membranes or other tissue. In some embodiments, the tip enclosure 220, 220 is disposable. In general, a tip enclosure, an interchangeable head assembly enclosure, and a disposable optic and sampling component as used herein are considered interchangeable terms.

FIGS. 12A-12C depict coupling of the tip enclosures shown in FIG. 11 to the device of FIG. 10. In one embodiment, a power supply and other controls 230 may alternatively be disposed outside of the hand-held base, as shown in FIG. 13. In various embodiments, the controls 230 include an on/off switch, a camera control, an illumination control (including illumination intensity control), RGB color control, lens adjustment, and white balance adjustment. Other controls can include a wireless antenna and circuit for wireless communication of video data from the device, as well as other interface connections, such as USB, Firewire, analog audio and/or video, and other interfaces or controls as described herein.

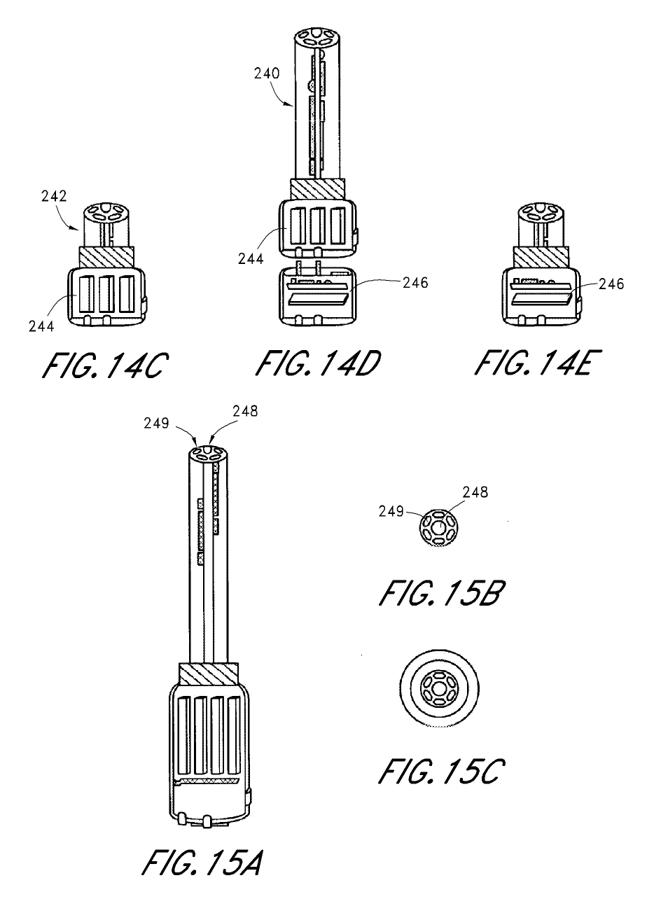

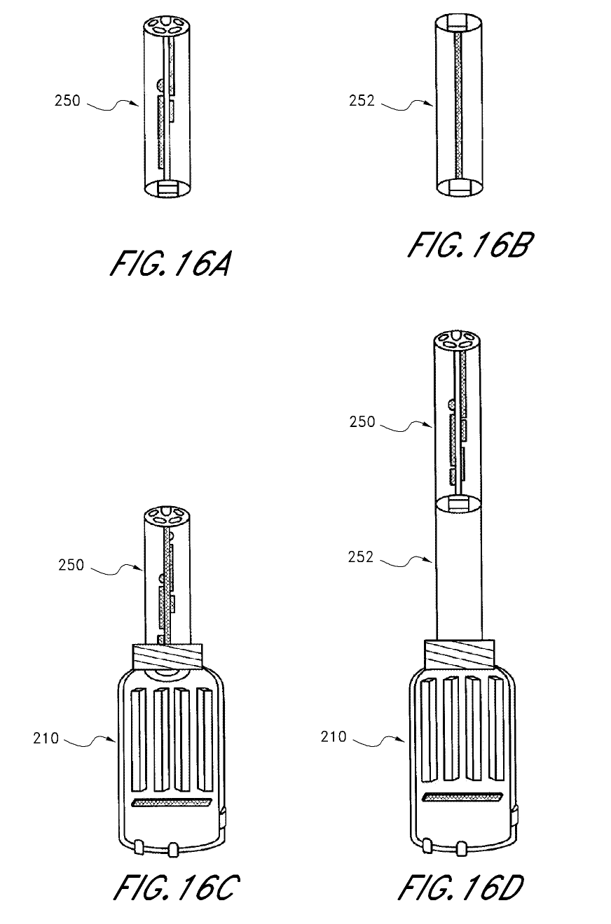

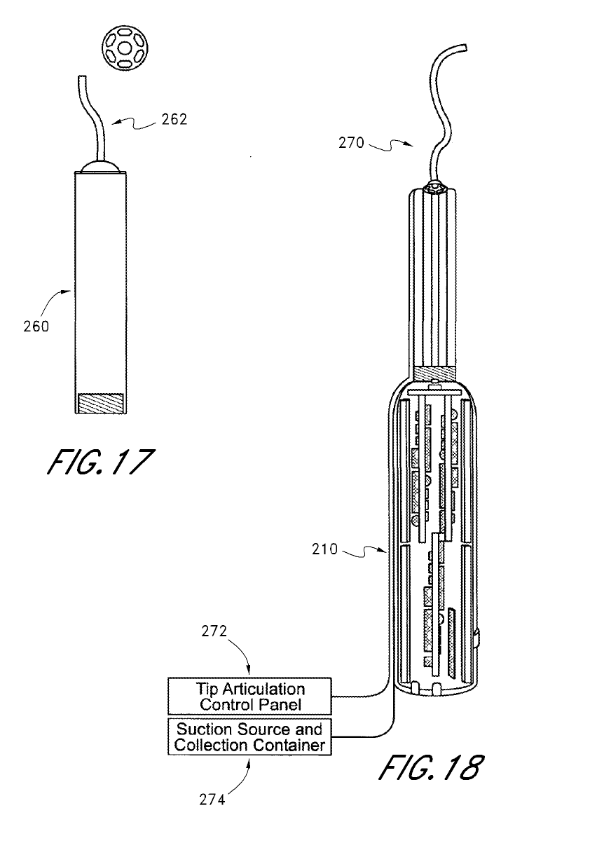

Exemplary interchangeable head assembly or tip configurations are depicted in FIGS. 14A-14E. The tip can include a tall extended lens 240 or short compressed lens 242. The tip may also include an internal battery controller 244 with a wireless transmitter 246. One embodiment with a tip configuration that includes a camera 248 and/or the light source 249, such as one or more LEDs, in the tip is depicted in FIGS. 15A-15C. An optional tip extender 252, having an extension shaft that extends the tip 250 (which can be the same or similar to interchangeable head assembly tips 212, 240, or 242) from a compressed mode to an extended mode, is shown in FIGS. 16A-16D.

As shown in FIG. 17, one embodiment of an interchangeable head assembly tip 260 includes a flexible fiber optic extension 262 through which illumination and/or the image is transferred via the tip to the camera in the hand-held base unit. The length of the fiber optic extension 262 depends on the particular application, and can range from one-half to twelve inches or more. The fiber optic extension 262 is illuminated by a lighting source on the tip of the camera, and fits snugly against the camera and light source so that no video return signal is lost or misdirected, and that all of the desired video is directed to the fiber optic extension 262. An exemplary device using a flexible tip 270 is depicted in FIG. 18. Preferably, the flexible tip 270 is actuated using an actuator 272 that is external to the tip (and, as illustrated, is external to the hand-held base 210), and in even more preferred aspects, the tip may further include a working tool such as, for example, a suction line or fluid line 274.

In various embodiments the lens at the distal end of the tip may have various optical properties. For example, the distal end of the tip can be shaped with a curvature that forms part of the lens. In such configurations, the distal end of the tip may have a central section that includes the optical fibers that transmit the reflected to and/or emitted light from the tip to the camera, while a plurality of circumferentially disposed sections may include optical fibers that provide the light from the light source to tissue being examined. In some embodiments, such tips provide a homogeneous illumination to the target area. In one embodiment a tip provides illumination to a target area, wherein the tip is small enough to enter passageways too small for larger lenses—i.e. through a cervix into a uterus. Where desired, a reflective or filtering coating may be applied to the distal end of the tip to reduce or even eliminate direct light transmission from the light source optical fibers to the camera optical fibers.

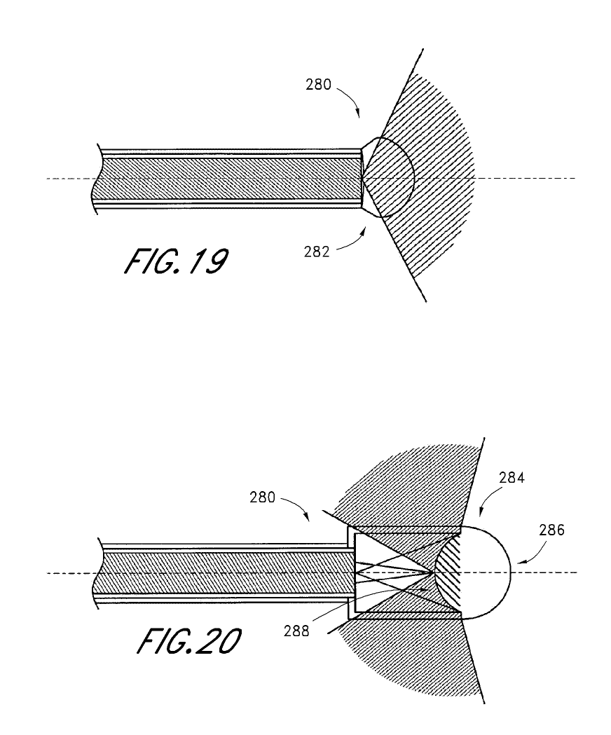

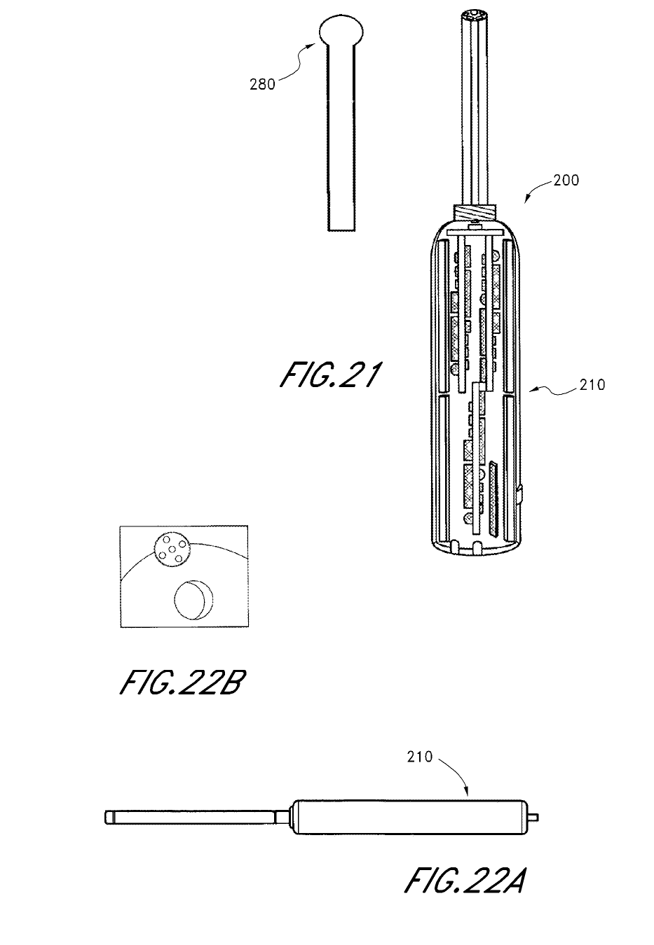

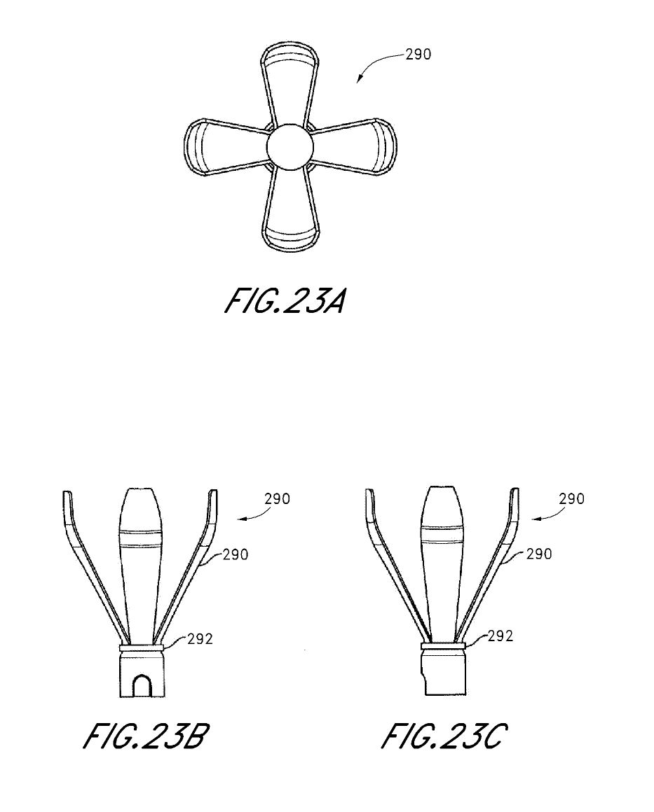

In one embodiment, it is also contemplated that the distal tip 280 of any of the interchangeable head assemblies (including any head assembly enclosures) described herein may be configured to provide a forward viewing tip 282 and/or an omni-directional viewing tip 284. Exemplary forward viewing tips 282 and omni-directional viewing tips 284 are depicted in FIGS. 19 and 20, respectively. Here, the forward viewing tip 282 has a front element with dual purpose: the front element provides a smooth spherical surface that acts as a tissue stabilizer or dilator which can spread the surrounding tissue reducing the patient discomfort, and acts as an optical element in front of the imaging optics to modify the image or increase the overall magnification. The front element of a forward-viewing tip 282 can be configured as a part of the imaging system to provide a microscope type of magnification and resolution at the near focus. The omni-directional viewing tip 284 may be configured to integrate all the previously described aspects of the forward viewing tip 282 on its distal end 286, while the rear surface 288 of the element is an aspheric surface designed to eliminate the back reflections from the LEDs into the field of view. As illustrated in FIG. 20, light emitted from a light source directed toward the distal end 280 of an interchangeable head assembly can reflect off the rear surface 288 to avoid potential glare interfering with the camera image. In an alternate embodiment, the omni-directional viewing tip 284 may be configured with a rear surface 288 which is reflective enough to act as a concave mirror for an axially oriented camera to view a wide viewing angle of the tissue to the side of the tip.

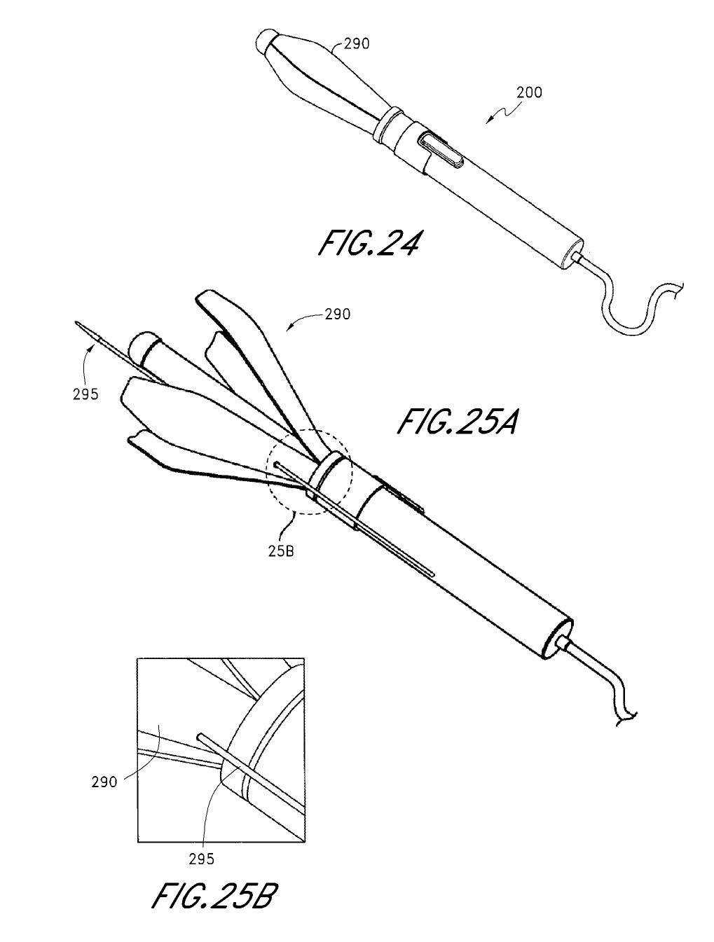

Exemplary aspects of such tips or tip enclosures 280 for use with an endoscopic device 200 are depicted in an exploded view in FIGS. 21 and 22. In one embodiment, the tip 280 is an omni-directional viewing tip 284 with an outer tube with a clear acrylic cylinder and a spherical front element to provide an omni-directional view of the surrounding walls or tissue. Here, the front element is part of the imaging system to provide a 360 degree peripheral field of view. The front surface of the element is spherical and acts as a tissue dilator or stabilizer, while the rear surface of the element can be designed as the one or several aspheric refractive or reflective surfaces to provide a distortion-free imaging of the surrounding tissue. The resulting image is then in a shape of a donut and can be viewed on a monitor or display screen. However, such an image can also be unfolded into a panoramic continuous flat image using software.

Certain embodiments of the interchangeable head assembly tips are configured such that the tip provides sufficient optical contact between the light guides for the camera and/or light source of the tip and the camera and/or light source that are located in the hand-held base. For example, the tip may be screwed into place using the proximal end of the tip. Alternatively, a bayonet lock or other temporary fastener may be employed to secure the tip to the hand-held base. The tip and the fastening mechanism are fabricated from material that can be sterilized and which allow coupling of the tip to the hand-held base. Accordingly, the base may include a disposable sterile cover. In such configurations, the device can be repeatedly used without undergoing sterilization by providing a disposable sterile cover to the base and a sterile disposable tip. Alternatively, the entire device may be covered by a disposable sterile cover. Further, the tip may be at least partially covered by a sterile and disposable sheath that provides magnification or other optical properties (e.g., modified viewing angle, etc.).

In certain embodiments, the hand-held base unit 210 preferably has a maximum length of less than fifteen inches, more typically less than ten inches, and preferably less than eight inches. Similarly, the diameter (or maximum width) of the hand-held base is less than three inches, more typically less than two inches, and preferably ranging from 0.5 to one inch in width. Suitable tips can have a length of between about 0.1 inches to 10 inches, and even longer. However, it is preferred that a length of the tip is between about 0.5 inches to 4 inches. Thus, as viewed in terms of a three-part device, the camera and/or light source of the device is disposed in the central third, while the distal third includes the disposable tip and the proximal third includes the hand-held base.

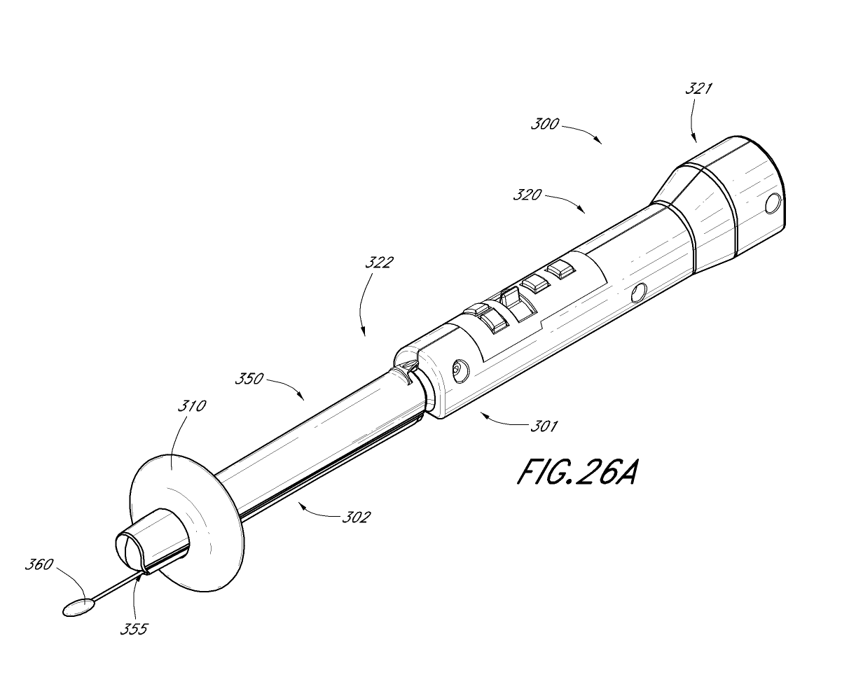

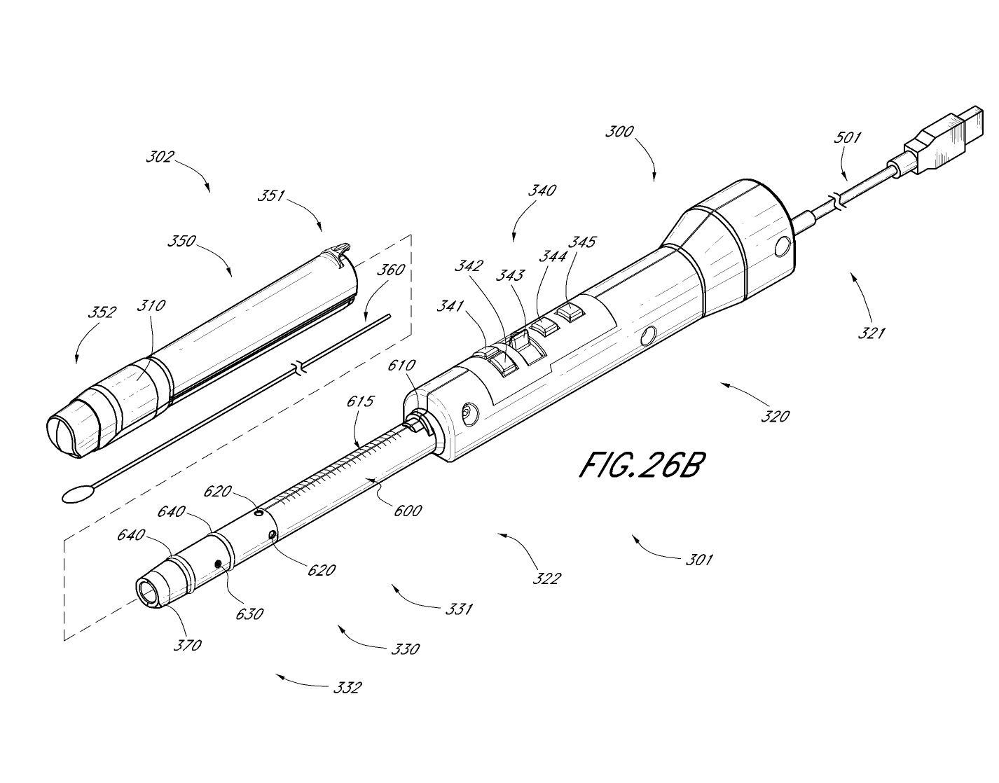

Additionally, or alternatively, contemplated interchangeable head assembly tips may also include a tissue stabilizer such as a mechanical tissue dilator 290. An exemplary dilator 290, which may be integral to the tip or removably attached to the tip, is depicted in FIGS. 23-25. A control ring 292 locks the dilator elements in a closed position, and the dilator elements spread out when the ring 292 is moved towards the base unit element. FIG. 24 shows the dilator 290 of FIG. 23 coupled to an exemplary device 200, wherein the dilator 290 is in a closed configuration. Where desired, additional working tools 295 (which may be any of the working tools described herein) may not only be located in the tip, but also within a space that is defined between the tip and the dilator arms, wherein the working tools 295 are preferably movable as depicted in FIG. 25.

### 3. Inflatable Tissue Stabilizer Embodiments

Disclosed further herein is an endoscopic device with embodiments configured with a base unit and an interchangeable head assembly comprising an inflatable tissue stabilizer, or a stabilizing ring. The embodiments of systems, devices, and methods discussed in this section have similar features and functionality to the systems, devices and methods disclosed elsewhere in this patent specification, except for the different or additional features as disclosed in further detail below. For example, embodiments of the endoscopic device 300 described herein include a base unit and an interchangeable head assembly. Various embodiments of the base unit and the interchangeable head assembly have features that are the same or similar to sizes, configurations, and functionality unless otherwise described below. Various embodiments of the interchangeable head assembly will include a tip enclosure that fits over at least a portion of the outer surface of the interchangeable head assembly. As used herein, the tip enclosure is considered a removable part or shell enclosing at least a part of the interchangeable head assembly. References to the interchangeable head assembly may include the interchangeable head assembly alone, or in conjunction with the tip assembly, depending on the context.

In one embodiment, the endoscopic device is comprised of a main probe and a tip enclosure. As used herein, the “main probe” may also be called an advanced sampling device (“ASD”), and comprises embodiments of a base unit and an interchangeable head assembly exclusive of the tip enclosure. Thus, in the context of describing a main probe, the interchangeable head assembly refers to interchangeable head assembly components exclusive of the tip enclosure. One reason for this distinction in this context is to distinguish between “permanent” parts of the endoscopic device from the “disposable” tip enclosure. As described above outside the context of a main probe, other embodiments of the tip enclosure could also be considered a part of an interchangeable head assembly—but for purposes of distinguishing the “main probe” or “ASD” from the tip enclosure, in certain embodiments the tip enclosure is a separate part. In any embodiment, the tip enclosure can also be called a “disposable optics and sampling component” (“DOSC”).

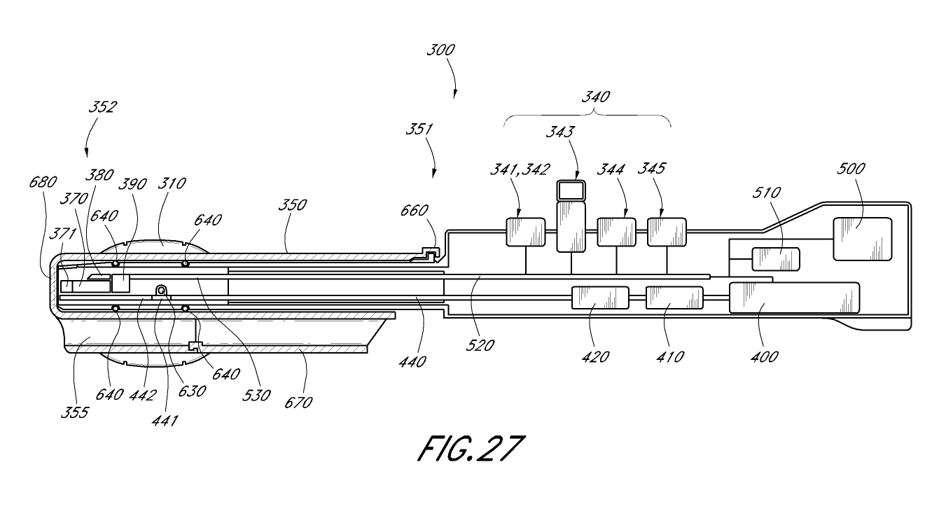

FIG. 26A illustrates a schematic, perspective view of an endoscopic device 300 with an inflatable tissue stabilizer 310 according to one embodiment of the present invention. In certain embodiments, the endoscopic device 300 has many of the features and functionality of the endoscopic devices 100 and 200 previously described herein. However, instead of comprising a tissue stabilization system with mechanical arms, certain embodiments of the endoscopic device 300 comprise an inflatable tissue stabilizer 310. As used herein, “tissue stabilizer” and “stabilizing ring” are interchangeable. The tissue stabilizer 310 is capable of dilating tissue in a manner similar to the mechanical dilators described above; however, it is also capable of finely controlled and measured stabilization of tissue within the bodily orifice. Relatively large dilation of tissue is not necessary for the inflatable tissue stabilizer 310 to be functionally effective, therefore one advantage of a controllable and measurable stabilization system is a reduction in discomfort and less of a risk of stretching or tearing tissue within the bodily orifice. In some embodied uses of the endoscopic device 300 the inflatable tissue stabilizer 310 does not need to be inflated, and is used optionally to stabilize and/or dilate tissue. In most preferred embodiments, an inflatable tissue stabilizer 310 is disposed on an outer surface of the tip enclosure 350, which is a part of the interchangeable head assembly 330. Although in some embodiments the tip enclosure 350 is a part of the interchangeable head assembly 330, the tip enclosure 350 is removable from the interchangeable head assembly 330. In certain embodiments without a tip enclosure, the inflatable tissue stabilizer 310 may be directly attached to the exterior surface of the interchangeable head assembly 330. However, as illustrated in a preferred embodiment, the inflatable tissue stabilizer 310 is attached to the exterior surface of the tip enclosure 350 at or near the distal end 353 of the tip enclosure 350.

One embodiment of the endoscopic device 300 comprises a main probe 301 and a tip enclosure 302, where the main probe 301 is also called an ASD, and the tip enclosure 302 is also called a DOSC. In one embodiment of tip enclosure 302, an inflatable tissue stabilizer 310 is disposed thereon.

Additional embodiments of the endoscopic device 300 also comprise one or more working tools 360. In certain embodiments, and as illustrated, the tip enclosure 350 has a working channel 355 for housing or transporting one or more working tools 360. The working tool 360 can be any of the variety of working tools disclosed herein.

FIG. 26B illustrates a schematic, partially exploded perspective view of the endoscopic device 300 of FIG. 26A according to one embodiment of the present invention. As illustrated, the endoscopic device 300 comprises a base unit 320 and an interchangeable head assembly 330. The base unit 320, which may also be called a control unit or handle, is sized and configured to be held in a human hand to permit functional and directional control of the endoscopic device 300. The base unit 320 has a proximal end 321 and a distal end 322. The interchangeable head assembly 330, which may also be called a head, tip, or interchangeable insertion unit, is sized and configured to be inserted into a bodily orifice, with the interchangeable head assembly 330 being removably connectable to the distal end 322 of the base unit 320, wherein the interchangeable head assembly 330 is detachably linked to the base unit 320 in one or more of either a mechanical, electrical, optical or fluid fashion. In one embodiment the endoscopic device 300 is comprised of a base unit 320 and an interchangeable head assembly 330 with a lens 370.

In one embodiment, the base unit 320 comprises one or more controls 340 for controlling various functional features of the endoscopic device 300. In various embodiments, the controls 340 may include any buttons, rocker switches, dials, joysticks, touchpads, slidable controls, or other control mechanisms. Each of the controls 340 is at least electrically, digitally, or mechanically connected to a control processor, hub, or functional device associated with at least one feature of the endoscopic device 300. As illustrated, the base unit 320 has controls 340 including an inflation control 341, a deflation and suction control 342, a focus control 343, an image zoom control 344, and an image capture control 345. Additional details relating to the functionality and components associated with these controls are discussed further below. In other embodiments, the controls 340 can be rearranged with additional or different controls which may be used depending on the features of the embodiment of the device, such as is discussed below.

As illustrated in FIG. 26B, one embodiment of a base unit 320 disposed in an endoscopic device 300 comprises an information transmitter 501 for communication between the endoscopic device 300 and a display, storage, transmission, or analysis unit (not illustrated), such as but not limited to a computer, monitor, television, personal digital assistant, printer, phone, satellite connection or other means for communicating information locally or to facilities or medical specialists world-wide. In various embodiments, the information transmitter 501 is a connector or a communicator. The endoscopic device 300 can further include data interfaces for transmission of data, such as a CCD signal from a camera, and the video signal from an image processor can be output via the information transmitter 501. In one embodiment, the information transmitter 501 is a port interface for a jack or connection to a communication cable via a wired interface (USB, USB2.0, Firewire, or any other hardwired means). Data that is sensed, recorded, or transmitted from the image sensor 390 or any apparatus within the endoscopic device 300 can be transmitted through the information transmitter 501 to an external device, as is discussed with the previously disclosed connectors in this specification. In one embodiment, the connector is a wired power source capable of transmitting data and/or providing electrical power for the endoscopic device 300 to power the device or to charge rechargeable batteries in a power source (not illustrated here) in the endoscopic device 300. In one embodiment, an information transmitter is a wireless connector which uses a radio transmitter, radiofrequency, infrared, microwave, Bluetooth, or any other wireless communication format or media known in the art which can support wireless transfer of data, video, audio, and other relevant functional information, as is discuss further below. Certain embodiments of an information transmitter include both wired and wireless options and interconnections.

The interchangeable head assembly 330 is removably attachable to the distal end 322 of the base unit 320 and can any of the features previously listed among the various tips, heads and enclosures described above, such as with reference numbers 110, 212, 220, 222, 240, 242, 244, 246, 250, 252, 260, 270, 280. The location of the interface between the interchangeable head assembly 330 and the base unit 320 depends on the embodiment of an intermediate shaft 600. The intermediate shaft 600 can be removably attachable to both the interchangeable head assembly 330 and the base unit 320, or it can be permanently attached to either the interchangeable head assembly 330 or the base unit 320 and considered a portion of the permanently attached part. The interchangeable head assembly 330 has a proximal end 331 and a distal end 332. In embodiments in which the intermediate shaft is permanently attached to the interchangeable head assembly 330 the intermediate shaft is part of the proximal end 331 of the interchangeable head assembly 330. In embodiments in which the intermediate shaft is permanently attached to the base unit 320, such as is illustrated in FIG. 26B, the intermediate shaft is part of the distal end 322 of the base unit 320.

As illustrated, a intermediate shaft 600 can optionally be used as a spacer or as a tip extender such as the previously disclosed tip extender 252, having an extension shaft that extends the tip 250 (which can be the same or similar to interchangeable head assembly tips 212, 240, or 242) from a compressed mode to an extended mode, as is shown in FIGS. 16A-16D. In embodiments comprising an endoscopic device 300 with an intermediate shaft 600, the location of the interface between the interchangeable head assembly 330 and the base unit 320 depends on the embodiment of the intermediate shaft 600. The intermediate shaft can be removably attachable to both the interchangeable head assembly 330 and the base unit 320, or it can be permanently attached to either the interchangeable head assembly 330 or the base unit 320. The interchangeable head assembly 330 has a proximal end 331 and a distal end 332. In embodiments in which the intermediate shaft 600 is permanently attached to the interchangeable head assembly 330 the intermediate shaft 600 is part of the proximal end 331 of the interchangeable head assembly 330. In embodiments in which the intermediate shaft 600 is permanently attached to the base unit 320, as is illustrated here, the intermediate shaft 600 is part of the distal end 322 of the base unit 320.

As illustrated, one embodiment of the interchangeable head assembly 330 has a removable outer surface comprising a removable tip enclosure 350, which may also be called a tip cover, a removably attachable interchangeable head assembly enclosure or a disposable optics and sampling component (“DOSC”). In one embodiment, the tip enclosure 350 is the same as tip enclosure 302. The tip enclosure 350 has a proximal end 351 and a distal end 352. In preferred embodiments, the distal end 322 of the base unit 320 terminates at or near the junction of the proximal end 351 of the tip enclosure 350. In other embodiments the distal end 322 of the base unit 320 extends within the interchangeable head assembly 330. Tip enclosure 350 may have many similar features, functionality and aspects as tip enclosures 220, 222 and 302, as described above. In certain embodiments, at least the distal end 352 of the tip enclosure 350 is at least partially transparent or capable of permitting the light source, image sensor or other sensor to properly interact or make a reading of the surrounding tissue in the bodily orifice. In one embodiment the tip enclosure 350 is an illumination diffuser. In one embodiment, at least a portion of the tip enclosure is configured with a material, panel, port, or coating to permit sensor readings, such as for an ultrasonic transducer or temperature sensor.

In one embodiment, a tip enclosure interface 610 is provided on or near the distal end of the base unit 320. In another embodiment, a tip enclosure interface 610 is provided on the intermediate shaft 600. The tip enclosure interface 610 provides a mechanical attachment for the tip enclosure (not illustrated here) to removably attach to and optionally lock or snap-fit to the rest of the endoscopic device 300.

In one embodiment, a dimensional indicator 615 is located on the endoscopic device 300 to assist a user in visually determining length, depth of insertion, rotation angles, and/or other dimensions for measurement of placement, orientation, or movement of the endoscopic device 300. The dimensional indicator 615 can be used to measure precise locations of abnormalities or anatomical features within the bodily orifice. In one embodiment, the dimensional indicator 615 comprises a ruler with a series of hatch marks and labeled numbers to indicate length in inches, centimeters, millimeters, or some other dimension in order to visually determine the depth of insertion of the endoscopic device 300 into a bodily cavity. Other embodiments of a dimension indicator (not illustrated here) include angles, etc. In certain embodiments in which the dimensional indicator 615 is located on the interchangeable head assembly 330, at least part of the tip enclosure is transparent in order to allow a user to see the dimensional indicator 615. In one embodiment, not illustrated, a dimensional indicator is provided on the exterior of a tip enclosure.

In one embodiment, the interchangeable head assembly 330 is removably attached to the intermediate section 600 with one or more fasteners 620. In certain embodiments, the fastener 620 is a screw, rivet, clamp, suction, or snap-fit. In other embodiments, the head assembly 330 is permanently attached to the intermediate section 600 by bonding, welding, or fabrication of the respective elements from a common medium.

In one embodiment, a balloon port 630 is provided in fluid communication with a internal channel or air channel (not illustrated here) that is in fluid communication with a pump system as described further below. The balloon port 630 is configured to work in conjunction with an inflatable tissue stabilizer 310, as has been described herein. The inflatable tissue stabilizer 310 can optionally be attached directly to the interchangeable head assembly or be attached to a tip enclosure 350 as is illustrated here. When the inflatable tissue stabilizer 310 is used in conjunction with a tip enclosure 350, one or more seals 640 provide a seal between the inflatable tissue stabilizer 310 and the tip enclosure in order to create a pressure differential for expressing or aspirating fluid media. In various embodiments, the seal 640 is an O-ring or a probe air gasket. In other embodiments, not illustrated here, an air or fluid port may be placed at an alternate location on the interchangeable head assembly 330 to provide, irrigate, or deliver a fluid to the bodily orifice, or to take a fluidic sample from the bodily orifice through the fluidic channel in the endoscopic device 300.

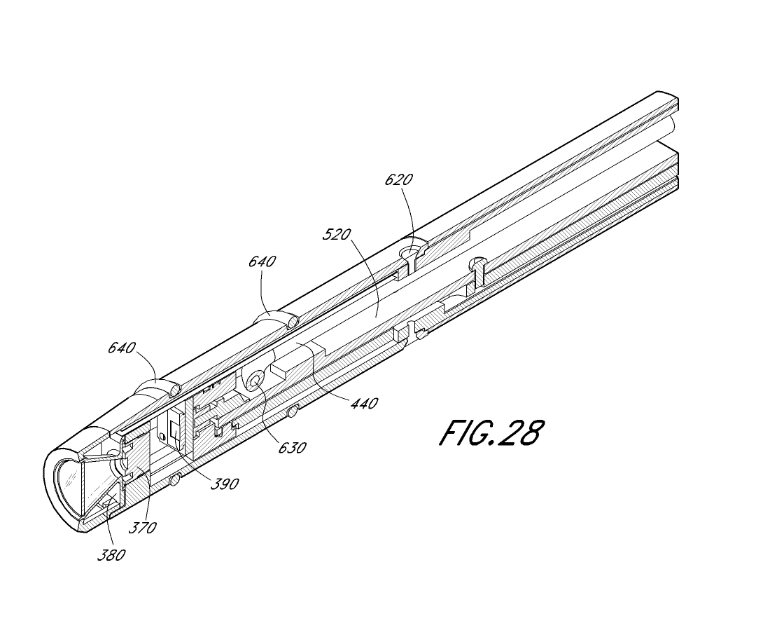

FIG. 27 illustrates a schematic cross-section view of the endoscopic device 300 of FIG. 26A according to one embodiment of the present invention, with the tip enclosure 350 attached to the main probe 301. One embodiment of the endoscopic device 300 comprises one or more master processing boards 520, 530. Board 530 is similar to master processing board 520 and can have the same or similar components, and in certain embodiments has specialized functionality related to the interchangeable head assembly embodiment. In one embodiment, board 530 is detachable from master processing board 520. In another embodiment board 530 is the same as or a part of master processing board 520. In one embodiment, the master processing board 520 comprises numerous digital and/or analog electronic components, including but not limited to one or more digital processors, image processors, microprocessors, central processing units (CPU), surface mount technology (SMT), surface mount devices (SMD), resistors, capacitors, connectors, diodes, or other electronic components which can be used as needed to regulate the current and digital data flowing across the circuit and to perform or control various functional features of the device. In various embodiments, the master processing board 520 is similar to other circuit embodiments described above. The master processing board 520 is electrically (or digitally) connected to the controls 340, other optional circuits or embodiments with additional sub-circuits, electrical components (such as will be described further herein), and electrical power supplied from a power source 510. In one embodiment, the power source 510 is a rechargeable battery. It is readily apparent to a person skilled in the art that there are many circuit variations possible for enabling and controlling an endoscopic device without departing from the spirit and scope of the invention as described herein. In one embodiment, the master processing board 520 is a single board extending distally from near the image sensor 640 along the interior of the interchangeable head assembly 330 and into the base unit 320. In another embodiment, one or more master processing boards 520 are electrically interconnected through one or more connectors between the separable portions of the endoscopic device 300. For example, in one embodiment, the processing boards have specific functionality that is supported depending on what type of interchangeable head assembly, intermediate section, or base unit is being used. Numerous processing boards are capable of work individually or in collaboration with each other can be electrically connected when the various modular features of the endoscopic device are interconnected.

As illustrated in FIG. 27, one embodiment of a base unit 320 disposed in an endoscopic device 300 comprises an information transmitter 500 for communication between the endoscopic device 300 and a display, storage, transmission, or analysis unit (not illustrated), such as but not limited to a computer, monitor, television, personal digital assistant, printer, phone, satellite connection or other means for communicating information locally or to facilities or medical specialists world-wide. In various embodiments, the information transmitter 500 is a connector or a communicator, and in one embodiment the information transmitter 500 is an information transmitter 501 as described above. In one embodiment, the information transmitter 500 is a wireless connector which uses a radio transmitter, radiofrequency, infrared, microwave, Bluetooth, or any other wireless communication format or media known in the art which can support wireless transfer of data, video, audio, and other relevant functional information. Certain embodiments of an information transmitter 500 include both wired and wireless options and interconnections.

In one embodiment, a power source 510 is included in the base unit 320 comprising a rechargeable battery (Li-ion or otherwise). A power cable (not shown) or inductive charging device (not illustrated) may be used to recharge the rechargeable batteries.

In one embodiment, a locating system is electrically connected to the endoscopic device 300 which can be configured to automatically sound or activate an alarm, vibration, reminder, or send a communication that the portable endoscopic device 300 is being moved or removed from a specified region, working area, or distance from a work station or office. In one embodiment, the automatic alarm system can be configured to work with the information transmitter 500.

As illustrated in FIG. 27, one embodiment of a base unit 320 disposed in an endoscopic device 300 comprises at least one of the group consisting of a pump 400, a check valve 410 and a solenoid valve 420—all of which are connected by an internal channel or air channel 440 in fluid communication with the inflatable tissue stabilizer 310 through a balloon port 630. The air channel 440 may also be called an internal channel, air channel, or air line. The inflation control 341 and deflation control 342 (also called a suction control) are in electrical communication with at least one of the group consisting of a the pump 400, check valve 410 and solenoid valve 420 in order to safely inflate or deflate the inflatable tissue stabilizer 310. In various embodiments, the pump 400 can be an air pump or a fluid pump which is configured to provide air or some other medium for inflating and suction for deflating a tissue stabilizer or stabilizing ring. In a preferred embodiment, pump 400 is an air pump with an optional air filter (not illustrated here).

In one embodiment, an air channel valve 441 is provided anywhere along the air channel 400 for selectively directing air pressure provided by the pump 400 between fluidic connectivity with the inflatable tissue stabilizer 310 and one or more functional fluidic devices 442. In one embodiment, as illustrated, the air channel valve 441 is provided at the distal end of the air channel 400. In one embodiment, the air channel valve 441 is controlled by an air channel valve controller (not illustrated here) capable of toggling between the various outputs including the inflatable tissue stabilizer 310 and one or more functional fluidic devices 442. In various embodiments, one, two, three, four, or more fluidic devices 442 are located at or near the distal end of the endoscopic device 300 and is configurable to be housed within a tip enclosure or an interchangeable head assembly, and is configured to be in fluidic communication with the interior of the bodily orifice. If the fluidic device 442 is disposed within the interchangeable head assembly, certain embodiments of the tip enclosure 350 will have a port or other channel for permitting fluid connection between the fluidic device 442 and the bodily orifice. In one embodiment, the fluidic device 442 has a port to the interior of the bodily orifice that is configured to be sealed, opened, or operatively open- and closeable to a reservoir or channel located within the fluidic device 442. In another embodiment, the tip enclosure 350 has a port to the interior of the bodily orifice that is configured to be sealed, opened, or operatively open- and closeable to a reservoir or channel located within the fluidic device 442.

In various embodiments, a fluidic device 442 is used for expressing or irrigating air, fluids, contrasts, dyes, medications, lubricants, semen, eggs, fertilized eggs, birth control devices or other media, and are also capable of being configured for collecting fluid or tissue samples from a bodily orifice. For example, in one embodiment the air channel valve 441 puts a port located at or near the distal tip of the endoscopic device 300 in fluid connection with the air channel 400. When positive air flow is needed, the port can deliver a burst of air from the air pump 400 to clear mucous, blood, fluids, or contaminants from the view of the camera. Alternatively, a positive air flow can be used to palpitate or manipulate tissue or other material within the bodily orifice.

In one embodiment a fluidic device 442 is a fluid delivery cartridge that contains a reservoir of fluid that can be actuated by a plunger within the cartridge that is in fluid connectivity with the output of one of the air channel valve 441 output channels. The replaceable cartridge can be loaded at the distal tip of the endoscopic device 300 prior to insertion, or it can be loaded via a channel extending toward the proximal end of the endoscopic device, or via an external reloading tube. When an air channel valve controller actuates the air channel valve 441 to become in fluidic connectivity with the cartridge plunger, the air pump supplies positive or negative pressure (corresponding to inflation or deflation/suction states for the balloon, inflatable tissue stabilizer 310, or stabilizing ring, respectively) to the proximal side of the plunger. Air pressure can actuate the cartridge plunger to advance distally, thereby expressing a fluid out of the fluid reservoir in the cartridge. This fluid can be a drug, dye, medication, contrast, irrigating fluid, or any other fluid for delivery into the bodily orifice. In a similar embodiment, a plunger may be part of the structure of the fluid line distal to the air channel valve 441 but still within the endoscopic device 300 instead of, or in addition to, the cartridge plunger described above. Likewise, in another embodiment, a fluid device 442 is a sample cartridge which can be loaded into the distal tip of the endoscopic device 300 and be placed in fluidic connectivity with the pump 400 via proper selection of the air channel valve 441. An empty reservoir can be configured to operate in a manner similar to a pipette, wherein a negative pressure or suction either works directly though suction in fluid connection with the reservoir in the sample cartridge, or works indirectly through a plunger which is advanced proximally in order to create suction at the sample cartridge interface port at or near the distal end of the endoscopic device.

In another embodiment, fluidic devices 442 or cartridges can be actuated by one or more manually or electrically controlled solenoids, lead screws, linkages, slidable shafts, or other means for moving a plunger to create an outward or inward flow of media in or out of the endoscopic device 300.

In FIG. 27, one embodiment of a check valve 410 is configured to limit the pressure or flow rate of the inflatable tissue stabilizer 310 or whatever medium is being transported in or out of the endoscopic device 300 by pressure in the air channel 440. One embodiment of a solenoid valve 420 is configured to limit the pressure or flow rate of the inflatable tissue stabilizer 310 or whatever medium is being transported in or out of the endoscopic device 300 by pressure in the air channel 440. In various embodiments, these components may be used to control air flow, liquid flow, gas flow or any number of gas or fluid media for controlling the actuation of the inflatable tissue stabilizer 310 or for interaction with orifice tissues for sampling, visualization, testing, or other purposes as described herein. In various embodiments, one or more among the pump 400, check valve 410 and solenoid valve 420 may be used in conjunction with an air pressure monitor, digital pressure monitor or other components to monitor media pressure to ensure the precise amount of media necessary to perform a procedure or task is used. Furthermore, the pressure monitor can provide a threshold pressure point, limit, safety zone, or range for the check valve 410 or solenoid valve 420 to ensure that the medium does not cause over expansion of the inflatable tissue stabilizer 310 or excess pressure or fluid flow speed of an expressed or sampled medium. Additional embodiments of this type of system are described below.

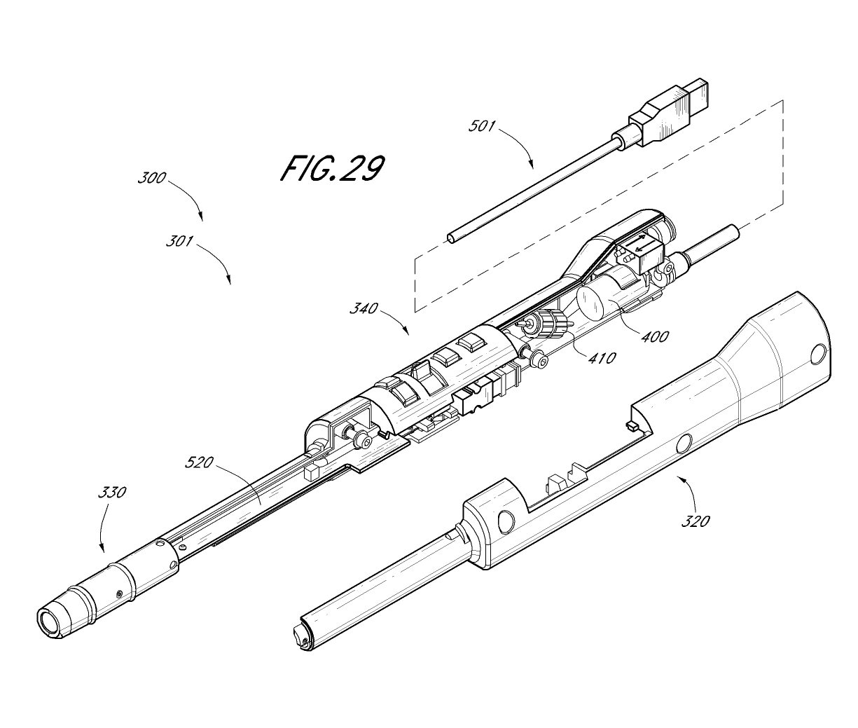

In the illustrated schematic embodiment in FIG. 27 and close up of a more detailed embodiment in FIG. 28, a lens 370, light source 380 and image sensor 390 are disposed within the interchangeable head assembly 330. These elements are located within the structure of the interchangeable head assembly 330 within the tip enclosure 350. Various embodiments of the lens 370, the light source 380, and the image sensor 390 are similar to the similarly named devices described above. Various embodiments of the lens 370 enhance a viewable image. Various embodiments of the light source 380 illuminate desired portions of the tissue. Various embodiments of the image sensor 390 include a camera, CCD, or video device. The image sensor 390 can be any of the variety of embodiments of image sensors disclosed within the present specification, including cameras, video systems, CCD'S, Doppler, ultrasound, and high-resolution imaging systems.

In one embodiment, the lens 370 is located distal to the image sensor 390. As described above, the lens 370 can be configured to magnify and/or focus an image. As illustrated in FIG. 28, the lens 370 can be housed in a module or baffle assembly which can be mechanically or digitally actuated to slide, rotate, and/or move to change how an image is transmitted to the image sensor 390. In various embodiments, an image can be altered with a targeting grid 680, which can be disposed on the lens 370 or on a distal tip portion of the tip enclosure 350 as is illustrated in FIG. 27. In various embodiments, the targeting grid 680 comprises a circle, a cross-hatch pattern, a square, a rectangle, a series of concentric circles, a grid of perpendicular crossing lines, or any other pattern which would assist in the analysis or measurement of an image within the bodily orifice. In another embodiment, the targeting grid is not physically manifested but is instead digitally interposed on an image electronically, such as by using software. In one embodiment, a digitally interposed targeting grid image of tissue or a feature from a previous examination, a measurement, a standard size or shape, or an image of an abnormality can be superimposed on the display image for analysis purposes.

In one embodiment, the light source 380 is at least one light-emitting diode (LED) configured to illuminate at least a portion of the bodily orifice. In one embodiment, one or more LEDs are located at the distal end 332 of the interchangeable head assembly 330 and surround the camera or CCD. In one embodiment the light source 380 is disposed on the same circuit board as an image sensor 390. In one embodiment, the light source 380 provides continuous illumination. In other embodiments, the light source 380 may be provided under pulse width modulation. With LEDs and certain other light sources, it is known in the art that pulse width modulation (PWM) can be used to change perceived brightness or intensity of the LED or create an imaging effect. For example, a LED is generally driven by micro-second flashes or bursts of either being “on” or “off”—depending on the duty cycle of the LED. Depending on the various embodiments being used, one or more LEDs or similar light sources may be pulsed in order to vary the intensity of the particular wavelength or wavelengths being emitted by the light source. In one embodiment, a light source, such as an LED, is pulsed to create a flash that is timed to coincide with a freeze-frame “photographic” image taken from the camera. The timing between a light source illumination period and an image-capture can be altered to account for delays between a flash and the capture of an image. In certain embodiments, a light source flash may be timed to illuminate for maximum illumination of an image capture by the camera, while in other embodiments, the timing is altered so that an image is captured after a period of maximum brightness or illumination in order to process the image more effectively. In one embodiment, a processor or micro-processor (discussed further below) controls pulses on the light source timing and illumination levels to enable higher resolution images without affecting the image sensor's automatic or reactive white balance or aperture, iris or timing adjustments. For example, in one embodiment, a LED light source is pulsed and allowed to diminish in illumination intensity before a camera captures an image. The delay allows the camera to capture image information at a higher resolution without reduction in aperture size or undesirable effects attributable to automatic white balance features in the camera.

As previously discussed within this specification, one embodiment of the lens 370, light source 380, and image sensor 390 may be configured to be physically movable with respect to each other in order to change focal lengths, focus, magnification, lighting features, and functional capabilities of the endoscopic device 300. Physical movement of various components can be accomplished using mechanical actuators that are manually moved with respect to each other, or by using solenoids, screws, or other mechanical actuators. In other embodiments, movement of elements can be controlled and changed electronically, physically, or both electronically and physically. In one embodiment of the present invention, actuation or activation of the lens 370, light source 380, and image sensor 390 are controllable with controls 340 located on the base unit 320. These controls 340 are electronically connected to the various components within the endoscopic device 300 in order to perform the respective functions. The tip enclosure 350 encloses at least part of the interchangeable head assembly 330 and has a working channel 355 for housing or actuating a working tool (not illustrated here). In one embodiment, the tip enclosure 350 is disposable.

FIG. 29 is a schematic, partially exploded perspective view of an embodiment of a main probe 301 of an endoscopic device 300 with a portion of the base unit 320 open to reveal the interior of the device. The reference numbers correspond to the description of parts in various embodiments discussed herein. In the illustrated embodiment of the endoscopic device 300, a connector is an embodiment of an information transmitter 501 that provides electrical power to the device in lieu of the presence of a separate power source (such as power source 510). In one embodiment, the connector is a wired power source capable of transmitting data and providing electrical power for the endoscopic device 300. In other embodiments, a power source (not illustrated), such as a rechargeable battery, is included.

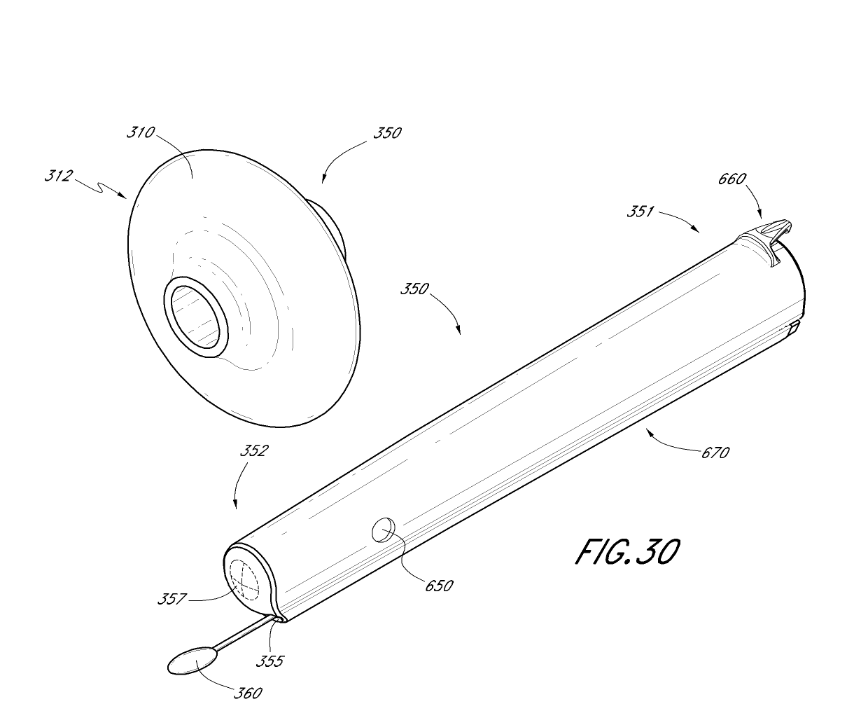

FIG. 30 illustrates a schematic, partially exploded perspective view of the tip enclosure 350 of the endoscopic device 300 of FIG. 26A with the inflatable tissue stabilizer 310 moved. Some embodiments of the tip enclosure 350 are similar in many ways to the other embodiments of tip enclosures described herein.

In one embodiment, the inflatable tissue stabilizer 310 is an expandable, flexible stabilizing ring which has many of the functional purposes as the previously disclosed arm-configured tissue stabilizers discussed herein. For example, the inflatable tissue stabilizer 310 can be inflated to hold back tissue that might be blocking or obscuring visualization of tissue within the bodily orifice, such as a wall of the vagina or a cervix or any other tissue. Furthermore, embodiments of the inflatable tissue stabilizer 310 are configured to facilitate the operation or extension of a working tool 360, such as a sample collection tool or a pap smear brush, which can be extended through a working channel 355 in the tip enclosure 350. In one embodiment, the inflatable tissue stabilizer 310 is sealed on its distal and proximal ends to the tip enclosure 350 in order to seal air, gas, fluid or some other substance within the inflatable tissue stabilizer 310. In one embodiment the seal is provided by bonding or adhesion. In another embodiment the seal is provide by the extension of the elastic, expandable material that comprises the inflatable tissue stabilizer 310 snuggly over the tip enclosure 350.

The fluid or gas medium inside the inflatable tissue stabilizer 310 can be augmented or removed in order to actuate a balloon-like inflation of the inflatable tissue stabilizer 310. The expansion of the inflatable tissue stabilizer 310 can be calibrated or limited such that when adequate pressure is reached to comfortably hold back tissue within the bodily orifice, the inflatable tissue stabilizer 310 material is compliant enough to start bending to maintain that pressure, while not overextending the tissue's expansion limits. Thus, discomfort by the subject, such as a mammal or patient, can be minimized. The inflatable tissue stabilizer 310 can be closed when the endoscopic device 300 is inserted, removed, or moved within the orifice. The inflatable tissue stabilizer 310 can be actuated to an enlarged configuration in order to hold the endoscopic device 300 in place within the orifice, or to position tissue for visualization or examination.

In one embodiment, a pressure monitor is fluidly connected to the pump, valve, or channel system in line with the inflatable tissue stabilizer 310. In one embodiment the pressure monitor is a sensor which is capable of accurately measuring, recording, and/or transmitting pressure data relating to a particular pressure that is needed to expand the inflatable tissue stabilizer 310 in a particular tissue region for a particular patient. For example, the flexibility of certain types of orifice tissue (such as a vagina, cervix, or other tissue) tends to decrease with age or certain medical conditions. Previous devices and methods for taking measurements of tissue flexibility or rigidity in the present art lack consistent standards because most practitioners tend to rely on manual palpitation or manipulation by a medical professionals hands, and is therefore highly subjective and difficult to accurately quantify or relate between measurements over time or between patients or between medical professionals. However, the pressure monitor can accurately and consistently measure the pressure in the inflatable tissue stabilizer 310 when it is applied to a tissue surface, and therefore allows for an accurate, consistent and useful measurement of important characteristics within the bodily orifice. For example, if the inflatable tissue stabilizer 310 is used in a particular location for a patient in order to monitor tissue rigidity or flexibility, a first reading from the pressure monitor may record a pressure of 6 psi, with subsequent measurements taken over time of 7 psi and 10 psi a medical practitioner could monitor changing rigidity or flexibility over time in order to make a diagnosis, etc.

The inflatable tissue stabilizer 310 can be used in conjunction with any of the visualization, monitoring, and working tool applications of the device as disclosed herein. For example, the inflatable tissue stabilizer 310 may be used to hold tissue in a particular orientation for an image or sensor based analysis of the tissue. In another example, a working tool 360 may be housed within the endoscopic device 300, or introduced through a channel or working channel within the endoscopic device 300. In various embodiments, the working tool 360 is actuated to perform its function once the inflatable tissue stabilizer 310 is expanded into place in order to stabilize the tissue of interest. Once the working tool 360 performs its function and is retracted back into the device or channel, the working tool 360 can be removed or replaced with a different working tool 360. The inflatable tissue stabilizer 310 can be deflated and the entire probe can be safely and comfortably removed from the orifice or cavity, and the working tool 360, which may be a collected sample in a shielded or unshielded collection device, can be removed from the working tool 360 for transport and analysis.

In the illustrated closed position, the inflatable tissue stabilizer 310 is deflated and fitted closely to the tip 300 to allow easy insertion and/or removal to and from the mammalian orifice or cavity. An internal channel, fluidic conduit, air line, or air channel (not illustrated here, but see air channel 440 in FIGS. 27 and 28) is fluidly connected to a pressure source, such as a manually-operated squeezable bulb, a syringe, a plunger, or the pump 400, check valve 410 and/or solenoid valve 420 sub-system described above. Air, fluid or other pressure-providing fluid enters the inflatable tissue stabilizer 310 driven by the pressure source via the fluidic conduit.

As has been previously disclosed with other embodiments of enclosures herein, one embodiment of the tip enclosure 350 is at least partially transparent to allow sensors or imaging devices located within the endoscopic device to function properly within a bodily orifice. Transparency of portions of the tip enclosure 350 (or illumination diffuser) permits the user to emit more light in selected areas adjacent or in proximity to a transparent region. Transparent portions of the tip enclosure 350 also allow a user to read a ruler or depth gauge (not illustrated here, but see the dimensional indicator 615 as shown in FIG. 26B) to visually determine length, depth of insertion, rotation angles, and/or other dimensions for measurement of placement, orientation, or movement of the endoscopic device.

In one embodiment of the tip enclosure 350 an enclosure port 650 is provided in the tip enclosure 350 which is in fluidic communication with the interior of an inflatable tissue stabilizer 310 and the balloon port 630 in the interchangeable head assembly 330, as described above. If the balloon port 630 roughly corresponds in location transversely with the enclosure port 650, the seals 640 as shown in FIG. 26B may be replaced with a single O-ring (not illustrated) circumferentially situated radially outwardly from the balloon port 630 to create a seal with the interior of the tip enclosure 350 to maintain the fluid integrity of a pressure level between the fluidic channel and the interior of the inflatable tissue stabilizer 310.

As illustrated in FIG. 30, a main body interface 660 is configured to interface with a tip enclosure interface (not illustrated here, but see the tip enclosure interface 610 as shown on FIG. 26B). In various embodiments, the main body interface 660 and tip enclosure interface 610 temporarily secure the tip enclosure 350 to either the base unit 320 or to another part of the interchangeable head assembly 330. As illustrated, the main body interface 660 is a cantilevered finger which snaps over a ridge located on the tip enclosure interface. In another embodiment, the finger may be located on the tip enclosure interface and a corresponding ridge may be presented on the main body interface 660. The interface can be a snap-fit, bayonet lock, rotational interlock, and can use a temporary or removable fastener, or utilize some other temporary fastening scheme that is known in the art.

In one embodiment of a tip enclosure 350, the tip enclosure 350 has a working channel cover 670 which axially extends along at least a portion of the working channel 355. As previously described, the working channel 355 accommodates a working tool 360 which can be advanced to perform a procedure within the bodily orifice. The working channel cover 670 is removably attachable, and can be deflected and snap-fit into place in order to proximally extend the working channel 355 from the distal end 352 of the tip enclosure 350. The working channel cover 670 can be removed to insert a working tool into the working channel 355, and replaced to secure the working tool while shielding the bodily orifice from the working tool. Alternatively, in some embodiments a working tool can be inserted into the working channel 355 without removing the working channel cover 670. The working channel cover 670 can be removed while the endoscopic device is inserted in the bodily orifice. In different embodiments the working channel cover 670 does not extend proximally to the proximal end 351 of the tip enclosure 350, but as illustrated, the working channel cover 670 extends proximally to the proximal end 351 of the tip enclosure 350.

In one embodiment of the tip enclosure 350 a tip enclosure lens 357 is provided for altering an image or light effect. The tip enclosure lens 357 can be configured in a similar manner as other lenses mentioned herein. In one embodiment, the tip enclosure lens 357 includes a targeting grid located proximally to the lens 370 or image sensor 390 of the endoscopic device 300. As illustrated, the targeting grid of the tip enclosure lens 680 comprises a circle and cross-hatch pattern which is etched into the distal end 352 of the tip enclosure 350. In other embodiments, the targeting grid of the tip enclosure lens 680 is a series of concentric circles, or a grid of perpendicular crossing lines, or any other pattern which would assist in the analysis or measurement of an image within the bodily orifice. In another embodiment, the targeting grid tip enclosure lens 680 is not physically manifested but is instead interposed on an image electronically, such as by using software. In one embodiment, the targeting grid of the tip enclosure lens 357 has similar features to the targeting grid 680 described above.

FIG. 30 also illustrates a schematic perspective view of an embodiment of a deflated inflatable tissue stabilizer 310. The inflatable tissue stabilizer 310 has a proximal end 311 and a distal end 312. In one embodiment the inflatable tissue stabilizer 310 is a stabilizing ring that extends circumferentially around or along a lateral surface to create a band or ring with an axis parallel to the longitudinal axis of the tip enclosure 350. In one embodiment the inflatable tissue stabilizer 310 is located at or near the distal end 352 of the tip enclosure 350. The proximal end 311 and distal end 312 of the inflatable tissue stabilizer 310 are at least partially sealed to a portion of the exterior surface of the tip enclosure 350 in order to at least temporarily maintain a pressure differential within the inflatable tissue stabilizer 310 in order to inflate or deflate the inflatable tissue stabilizer 310. The seals can be provided by bonding, adhesion, or other similar processes. The seals also prevent the inflatable tissue stabilizer 310 from sliding along the tip enclosure 350 during insertion, retraction, rotation, or any movement of the endoscopic device, such as within a bodily orifice.

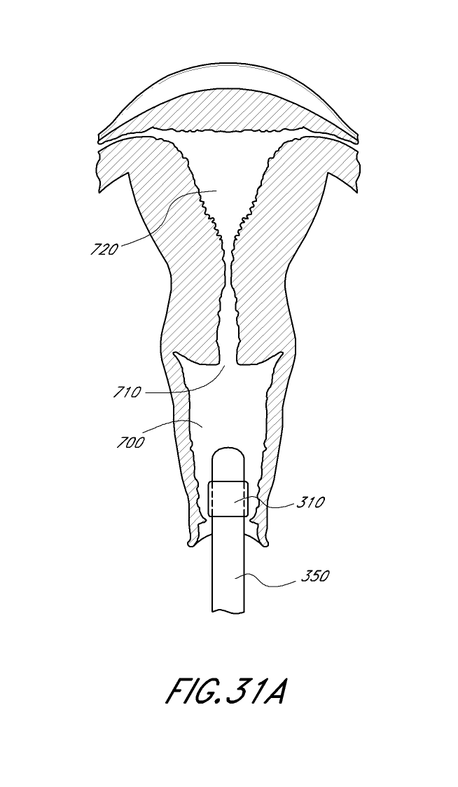

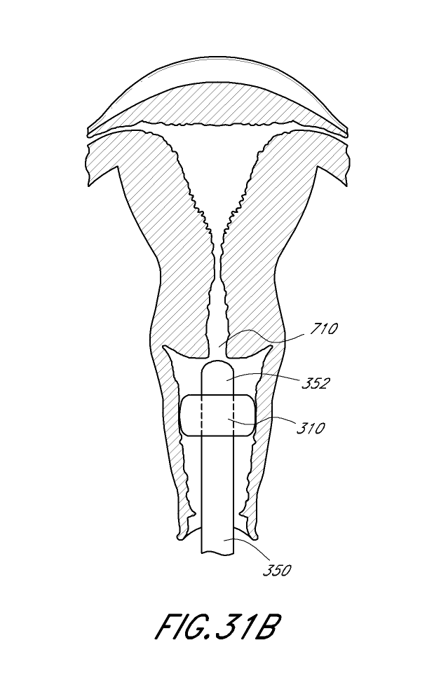

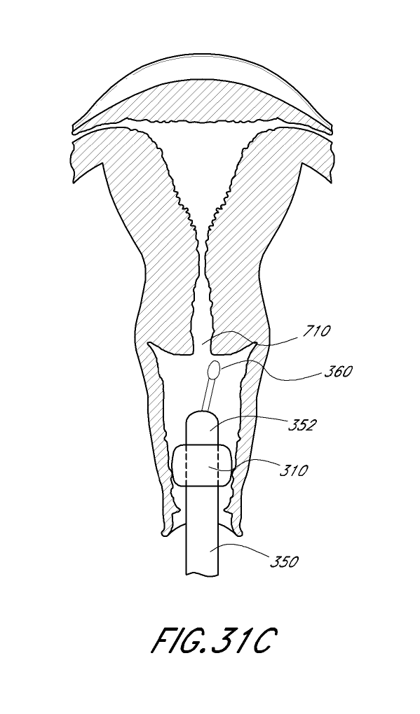

FIGS. 31A-31D illustrate some of the steps in one embodiment of a method for using any of the disclosed embodiments of an endoscopic device 300 with an inflatable tissue stabilizer 310 disposed on a tip enclosure 350 with an optional exemplary working tool 360. In one embodiment the endoscopic device 300 is comprised of a main probe and a tip enclosure 350. The main probe, or ASD, is comprised of a base unit 320 and an interchangeable head assembly 330. The base unit 320 is sized and configured to be held in a human hand to permit functional and directional control of the device. The interchangeable head assembly 330 is sized and configured to be inserted into a bodily orifice, such as a vagina 700 as illustrated in FIGS. 31A-31D. The distal end of the vagina 700 includes a cervix 710 which leads a uterus 720.

In one embodiment of a method of using the endoscopic device 300, a main probe 301 is provided. In one embodiment, an optional protective sheath (not illustrated here) may be wrapped or slipped over at least a portion of the main probe 301. The protective sheath may be made of a thin, flexible, material such as latex, and provide a disposable protective barrier to contamination of the main probe 301 in a manner similar to a surgical glove or condom. In one embodiment, the protective sheath is advanced over the interchangeable head assembly 320 and base unit 320, leaving an open proximal end proximal to the proximal end 321 of the base unit 320 for a connector to pass through. In one embodiment, the protective sheath has a distal port or opening to allow at least a portion of the interchangeable head assembly 320 to properly interact with a tip enclosure 350.

Prior to insertion into the bodily orifice of a subject, the tip enclosure 350 is placed over the distal end 332 of the interchangeable head assembly 330 and temporarily locked in place at an interface 610, 660 between the tip enclosure 350 and interchangeable head assembly 330. Before, after, or during the temporary locking of the interface 610, 660 an optional working tool 360 may be inserted into the working channel 355 of the tip enclosure 350. The distal opening of the protective sheath is covered by the tip enclosure 350 to prevent at least some contamination.

In various embodiments, the working channel cover 670 can remain in place or be removed and replaced while a working tool 360 is inserted proximally or distally into the working channel 355.

FIG. 31A illustrates a schematic side view of an embodiment of the insertion or removal of an endoscopic device 300 with a deflated inflatable tissue stabilizer 310 and an optional exemplary working tool 360 in a retracted position. As the endoscopic device 300 is inserted or removed from the bodily orifice it is advantageous to maintain the profile or configuration of the endoscopic device 300 as illustrated in FIG. 33A in order to prevent the inflatable tissue stabilizer 310 or working tool 360 from causing discomfort or pain in overcoming tissue obstructions while moving within the orifice. In the illustrated embodiment, the bodily orifice is a vagina 700. Various media can be used to coat the endoscopic device. For example, a lubricant, ultrasonic transduction gel, or other media for altering or improving image capture quality can be placed between the interchangeable head assembly 330 and the tip enclosure 350. Likewise, media can be placed on the exterior surface of the tip enclosure 350.

FIG. 31B illustrates a schematic side view of the endoscopic device 300 of FIG. 31A with an inflated inflatable tissue stabilizer 310. The endoscopic device 300 is manually directed by a user holding the base unit 320 to a region for examining, visualizing, diagnosing, manipulating, treating or recording of abnormalities with interior regions of body cavities. As discussed above, the base unit 320 has controls 340 including an inflation control 341, a deflation and suction control 342, a focus control 343, an image zoom control 344, and an image capture control 345. The user or operator actuates the respective control in order to perform the function for that control. All these controls 340 can be utilized when the inflatable tissue stabilizer 310 is deflated. Data or information can be transmitted from the endoscopic device to an image display device. For example, in one embodiment, transmitting information comprises capturing image data with an image sensor disposed within the interchangeable head assembly.

When the inflatable tissue stabilizer 310 is inflated to an open or radially expanded position, the inflatable tissue stabilizer 310 expands the orifice or cavity to push back tissue therein, and create a larger opening for the endoscopic device 300. Accordingly, a larger surface area of the orifice or cavity is in view of the distal end 332 of the interchangeable head assembly 330 of the endoscopic device 300, and an image sensor directed toward the distal end 332 will have a wider angle of view. In one embodiment, the inflatable tissue stabilizer 310 dilates a portion of the tissue within the orifice for improving visualization within the orifice, wherein the dilating comprises actuating a dilating assembly from a first configuration to a second configuration, wherein the second configuration has a second radial cross-sectional area defined by the outer surface of the dilating assembly that is greater than a first radial cross-sectional area defined by the outer surface of the dilating assembly in the first configuration. In one embodiment, the dilating assembly is an inflatable tissue stabilizer 310. As illustrated, the distal end of the tip enclosure 352 can be positioned in close proximity to the cervix 710.

FIG. 31C illustrates a schematic side view of the endoscopic device 300 of FIG. 31B with an inflated inflatable tissue stabilizer 310 and a deployed exemplary working tool 360. In one embodiment, a working tool 360 may be extended to interact with the bodily orifice tissue or material disposed therein after the inflatable tissue stabilizer 310 is inflated to create a stable working surface. In various embodiments, the relative position of the endoscopic device 300 and the location of the inflation of the inflatable tissue stabilizer 310 may be modified or altered as necessary for the purpose of the procedure. The angle of the working tool 360 with respect to the longitudinal axis of the endoscopic device 300 can be manually altered. Alternatively, in another embodiment, the working tool 360 does not require the inflatable tissue stabilizer 310 to be inflated in order to extend and actuate. In one embodiment, while the inflatable tissue stabilizer 310 is in a closed, or radially compact, or deflated state, there a potentially wider range of motion for the device operator to work with, and the working tool may be able to access regions of the bodily orifice that are less accessible if the inflatable tissue stabilizer 310 is inflated.

FIG. 31D illustrates a schematic side view of the endoscopic device 300 of FIG. 31B with an inflated inflatable tissue stabilizer 310 and a deployed flexible tip 271. In one embodiment, flexible tip 271 is similar to the flexible tip 270 as depicted in FIG. 18. In various embodiments, flexible tip 271 is a working tool 360 advanced through the working channel 355, or a type of steerable interchangeable head assembly. Various embodiments of the flexible tip 271 include a microcatheter, steering means, a fiber optic, a light tube, an image recording tool, manipulation tools, fluidic devices, reservoirs for deploying sperm or an egg or a contraceptive or other device or material, a pipette, sample reservoirs, and various miniaturized versions of embodiments of devices described herein. In other embodiments, the flexible tip 271 may be advanced into a nose, ear, anus, urethra, or other orifice.

Throughout this specification various embodiments of removably connectable working tools to assist in a diagnostic, therapeutic or anatomical assessment have been disclosed. In various embodiments, the working tool, such as working tool 295 or 360 is a laparoscopic device, a colposcopic device, a loop electrosurgical excision procedure (LEEP) device, a pap smear apparatus, a fluid line, a thermometer, a Doppler, an ultrasound emitter, a biopsy apparatus, a cutting device, a cauterizing device, needle, reservoir, fluidic device, fiber optic, or other device. Certain embodiments of working tools 360 include an elongate body for manually extending, manipulating, controlling, and retracting the working tool through a working channel such as working channel 355. A portion of the elongate body can be held as a handle for the working tool. In one embodiment of a method of using any of the embodiments of working tools 360, the steps comprise distally advancing one or more removably connectable working tools 360 within a working channel 355 of the endoscopic device 300, using the working tool 360 to assist in the diagnostic, therapeutic or anatomical assessment of tissue within the orifice, and proximally retracting the one or more removably connectable working tools 360.

For example, in one embodiment of a fluid line working tool (not illustrated), a tube is connected to a fluidic port (not illustrated) similar to balloon port 630 or fluidic device 442 port, as described above. Embodiments of the fluid line working tool can be in direct or indirect fluidic communication with an air channel (such as air channel 440 shown in FIG. 27). The fluidic port could be located in the interchangeable head assembly 330 or in the base unit 320. A portion of the fluid line could be flexible near the fluidic port and more rigid along a length of the fluid line in order to control, extend, and retract the fluid line within the working channel 355. The fluid line could also bifurcate into a fluid reservoir or sample collection device.

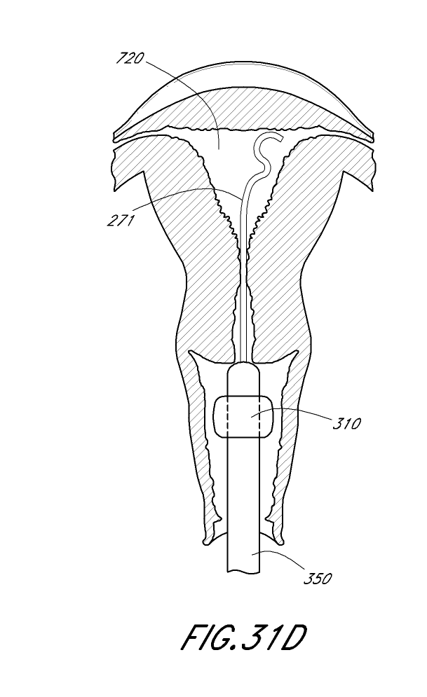

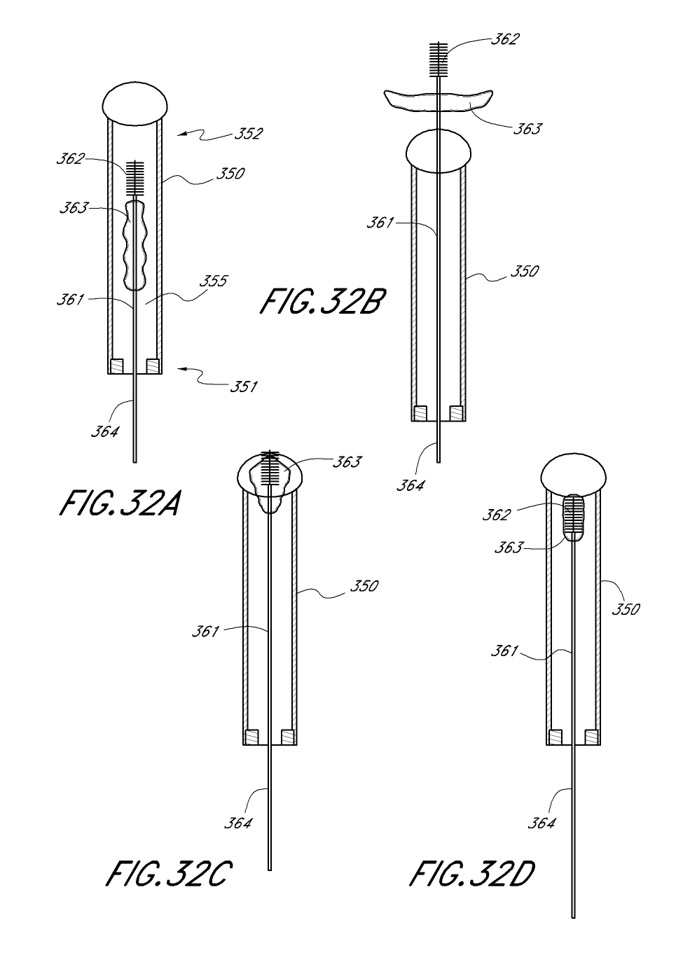

FIGS. 32A-32D illustrate a side schematic of an embodiment of a shielded working tool, where the shielded working tool is a sleeved sample collection device 361. As illustrated in FIGS. 32A-32D, an embodiment of a working channel 355 is shown in a sectional view revealing a removable embodiment of a working tool therein. Although the following description applies to an embodiment of the sleeved sample collection device 361, the concept can be applied to any working tool, whether the tool is for visualization, media or device delivery, manipulation, or any other function for a working tool. In general, working tools without a shield or protective sleeve run the risk of contamination while being moved between a target site within a bodily orifice and a laboratory for analysis. Potential sources of contamination include non-target site portions of the bodily orifice and even a working tool delivery system. The use of a working channel, such as working channel 355 described above provides an advantage of a channel relatively free of contamination for the working tool 360 to be moved within. However, the use of the same working channel 355 for multiple working tools 360 can lead to potential cross contamination of the tools. Whether a working tool is used in conjunction with a working channel (such as working channel 350) or not, the addition of a shield or sleeve to the working tool helps provide a protective layer against contamination.

In one embodiment, the sleeved sample collection device 361 is a pap smear brush. In one embodiment, a sleeved sample collection device 361 comprises an elongate body 364, a sample collection portion 362 at a distal end of the elongate body 364, and a shield 363 which is deployable over or proximal to the sample collection portion 362. In various embodiments, the elongate body 364 is a working tool handle, the sample collection portion 362 is a pap smear brush with bristles, and the shield 363 is a sleeve, mesh, barrier, or enclosure made of cloth, fabric, plastic (such as cellophane), flexible metal, shape-memory metal, or other flexible, pliable materials, composites or assemblies. In one embodiment, the shield 363 is a mesh biased to extend radially outward from the elongate body 364 in a location adjacent and/or proximal to the sample collection portion 362. If the sleeved sample collection device 361 is unloaded, the shield 363 extends radially outwardly from the elongate body 364 and the bristles of the sample collection portion 362 brush are extended outward in a manner similar to that illustrated in FIG. 32B. If the sleeved sample collection device 361 is loaded or compressed from the distal end of the working channel 355 or a tube, the walls of the working channel 355 collapse the shield 363 distally over the sample collection portion 362, thereby enclosing at least part or preferably all of the sample collection portion 362. See FIG. 32D. If the sleeved sample collection device 361 is loaded or compressed from the proximal end of the working channel 355 or a tube, the walls of the working channel 355 collapse the shield 363 proximally away from the sample collection portion 362, thereby exposing the sample collection portion 362 to the interior walls of the working channel 355 or tube while the sample collection device 361 is advanced to the distal end of the working channel 355 or tube. See FIG. 32A. When the sample collection device 361 is extended distally beyond the walls of the working channel 355 as illustrated in FIG. 32B, the radially outward bias in the shield 363 reverts the sample collection device 361 shield 363 to an open configuration, and the bristles of the pap smear brush are fully extended and the shield 363 is in a radially expanded configuration. A sample can be delivered or taken, and then the proximal withdrawal of the sample collection device 361 causes the shield 363 to wrap around the sample collection portion 362 or brush, and the walls of the working channel 355 collapse the shield 363 distally over the sample collection portion 362, thereby enclosing at least part or preferably all of the sample collection portion 362. See FIG. 32C.

In another embodiment, the sleeved sample collection device 361 also comprises a guidable tube or sleeve (not illustrated) with walls that actuate the shield 363 in a manner similar to the interior walls of the working channel 355 as described herein. While in transport, at least a distal portion of the elongate body 364, sample collection portion 362, and a shield 363 are located within a lumen of the tube. When the tube is withdrawn proximally with respect to the elongate body 364, sample collection portion 362 or shield 363, or when the elongate body 364, sample collection portion 362 or shield 363 are advanced distally with respect to the tube (or both), the sample collection portion 362 and shield 363 can be radially outwardly deployed or expanded to gather a sample or deliver a medium or device in the working tool.

In another sleeved sample collection device embodiment, the sleeve is an axially actuating cylinder, tube, or slide that encloses a sample collection portion in transit but allows the sample collection portion to become exposed to a target site, in a manner similar to that discussed above.

Although not presently illustrated, various other embodiments of devices, parts, and sub-assemblies have been contemplated for the present invention. For example, in some embodiments the controls 340 can be rearranged with additional or different controls which may be used depending on the features of the embodiment of the device. For example, controls configured to control other contemplated functions that are not presently illustrated in the illustrated embodiment include a temperature gauge and/or a thermometer for measuring specific tissue temperatures, which can be used to monitor or diagnose infections or other conditions. Materials to allow heat transfer and heat readings may be inserted In another contemplated embodiment, a heating element is provided within the endoscopic device to warm air or media to be expelled or samples that have been extracted from a bodily orifice at a controllable temperature, such as at or near body temperature. Additional contemplated embodiments include a Doppler system, an ultrasound system, and other imaging technologies that may be added to the endoscopic device 300 which will provide added benefits, such as density and depth of abnormality measurements or treatment or therapeutic uses. Additional contemplated functions also include using special or specific frequency light or lighting patterns, or other emerging technologies to heal afflictions or abnormalities naturally without need for medication. For example, phototherapy which has been shown to be effective in treating osteoarthritic pain and inflammation may also have beneficial therapeutic effects for treating infections or abnormalities within various bodily orifices.

Thus, specific embodiments and applications of multi-functional video scopes and endoscopes have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps can be present, or utilized, or combined with other elements, components, or steps that are not expressly referenced.

What is claimed is:

1. An endoscopic device for the examination of tissue within a corporeal orifice to permit diagnostic, therapeutic or anatomical assessment, comprising:
a base unit sized and configured to be held in a human hand to permit functional and directional control of the device, the base unit having a proximal end and a distal end;
an interchangeable head assembly sized and configured to be inserted into the orifice, the interchangeable head assembly being removably connectable to the distal end of the base unit, wherein the interchangeable head assembly is detachably linked to the base unit in one or more of either a mechanical, electrical, optical or fluid fashion; and
an inflatable tissue stabilizer disposed external to a distal end of the device.

2. The endoscopic device of claim 1, further comprising an image sensor.

3. The endoscopic device of claim 2, wherein the image sensor is disposed within the interchangeable head assembly.

4. The endoscopic device of claim 2, further comprising at least one lens for enhancing a viewable image.

5. The endoscopic device of claim 4, wherein the at least one lens is part of the interchangeable head assembly.

6. The endoscopic device of claim 2, further comprising a light source to illuminate desired portions of the tissue.

7. The endoscopic device of claim 6, wherein the light source comprises at least one light emitting diode (LED).

8. The endoscopic device of claim 1, further comprising an information transmitter for communication between the device and a display unit.

9. The endoscopic device of 27, wherein the information transmitter comprises a wireless communication device.

10. The endoscopic device of claim 1, further comprising an internal power source.

11. The endoscopic device of claim 1, further comprising one or more removably connectable working tools to assist in the diagnostic, therapeutic or anatomical assessment.

12. The endoscopic device of claim 11, wherein the working tool is selected from the group consisting of a laparoscopic device, a colposcopic device, a pap smear apparatus, a fluid line, a thermometer, a thermal sensor, a Doppler system, an ultrasound emitter, a biopsy apparatus, a cutting device, a cauterizing device, and a needle.

13. A method of examining tissue within a corporeal orifice, the method comprising:
inserting an endoscopic device having a base unit sized and configured to be held in a human hand to permit functional and directional control of the device, the base unit having a proximal end and a distal end and an interchangeable head assembly sized and configured to be inserted into the orifice, the interchangeable head assembly being removably connectable to the distal end of the base unit, wherein the interchangeable head assembly is detachably linked to the base unit in one or more of either a mechanical, electrical, optical or fluid fashion, and an inflatable tissue stabilizer disposed external to a distal end of the device; and
transmitting information from the endoscopic device to an image display device.

14. The method of claim 13, wherein transmitting information comprises capturing image data with an image sensor disposed within the interchangeable head assembly.

15. The method of claim 13, wherein transmitting information comprises wirelessly transmitting data using a wireless communication device.

16. The method of claim 13, further comprising manipulating a portion of the tissue within the orifice for improving visualization within the orifice, wherein the manipulating comprises actuating the inflatable tissue stabilizer from a first configuration to a second configuration, wherein the second configuration has a second radial cross-sectional area defined by the outer surface of the inflatable tissue stabilizer that is greater than a first radial cross-sectional area defined by the outer surface of the inflatable tissue stabilizer in the first configuration.

17. The method of claim 13, further comprising;
distally advancing one or more removably connectable working tools within a working channel of the endoscopic device;
using the working tool assist in the diagnostic, therapeutic or anatomical assessment of tissue within the orifice; and
proximally retracting the one or more removably connectable working tools.

18. An apparatus for the diagnostic, therapeutic or anatomical assessment of tissue within a vagina, comprising:
an interchangeable insertion unit configured to be removably coupled to a control unit in one or more of either a mechanical, electrical, optical or fluid fashion, the interchangeable insertion unit further sized and configured to be insertable within the vagina and to transmit data to an external data display device; and
a stabilizing ring disposed on a distal end of the interchangeable insertion unit.

19. The apparatus of claim 18, further comprising an image sensor.

20. The apparatus of claim 18, further comprising one or more removably connectable working tools to assist in the diagnostic, therapeutic or anatomical assessment.
